(19) United States
(12) Patent Application Publication
(10) Pub. No.: US 2025/0014344 A1
(43) Pub. Date: Jan. 9, 2025

(54) METHOD FOR GIVING FEEDBACK ON A SURGERY AND CORRESPONDING FEEDBACK SYSTEM

(71) Applicant: CARL ZEISS MEDITEC AG, Jena (DE)

(72) Inventors: Alexander FREYTAG, Erfurt (DE); Amelie KOCH, San Francisco, CA (US); Liesa BREITMOSER, München (DE); Euan THOMSON, Tiburon, CA (US); Dmitry ALPEEV, München (DE); Ghazal GHAZAEI, München (DE); Werner SCHAEFER, München (DE); Sandipan CHAKROBORTY

(21) Appl. No.: 18/684,402

(22) PCT Filed: Aug. 17, 2022
(86) PCT No.: PCT/EP2022/072933

§ 371 Date: Feb. 16, 2024

Related U.S. Application Data
(60) Provisional application No. 63/234,446, filed on Aug. 18, 2021.

(51) Int. Cl. G06V 20/40; G06T 7/00; G06V 10/25

(52) U.S. Cl. CPC G06V 20/49; G06T 7/0012; G06V 10/25; G06T 2207/30041

(57) ABSTRACT

Disclosed is a method for giving a feedback on a surgery, in particular an eye surgery, the feedback method comprising loading and/or receiving video data from a surgery, analyzing the video data, evaluating the analyzed video data, and outputting and/or displaying the evaluation result. Disclosed is further a feedback system for surgeries, in particular eye surgeries, the feedback system comprising a processing device for loading and/or receiving video data from a surgery, for analyzing the video data, and for evaluating the analyzed video data, and an output device for outputting and/or displaying the evaluation result.

### CROSS-REFERENCE TO RELATED APPLICATIONS

The present application is the U.S. National Stage entry of International Application No. PCT/EP2022/072933, filed Aug. 17, 2022, which claims priority under 35 U.S.C. § 119 to U.S. Provisional Patent Application No. 63/234,446, filed Aug. 18, 2021, the contents of which are incorporated by reference herein in their entirety.

### TECHNICAL FIELD

The present invention relates to a method for giving a feedback on a surgery. The present invention further relates to a corresponding feedback system.

### BACKGROUND

Surgeries, in particular eye surgeries like cataract surgeries, are extremely complicated and require high skills to ensure an optimal surgery outcome that meets the expectations of a patient, for example regarding the visual acuity or recovery period. Based on variations of the specific operation field, patient history, etc., surgeons need to know different surgery techniques, various tools, and need to be able to handle different complications during surgeries, etc. As a consequence, surgeons require intensive training before they become expert in the specific operating field.

In order to adopt a new technology, it is conjectured that a surgeon needs to physically carry out at least 200 operations. Further, the conducted operations need to be reviewed by an expert for receiving feedback which can help in analyzing own opportunities for improvement, e.g., with regard to tool handling, speed, alternative surgery techniques, microscope usage, total time-spent in the surgery, surgical efficiency, etc. Especially surgeons adopting a new technique or the ones who are generally in a learning curve receive too little and/or unstructured feedback from colleagues or mentors. Thus, an objective comparison to peers, to experts or mentors, to similar surgeries, or to the surgeons themself over time is difficult. Furthermore, feedback is often not immediately available, e.g., when mentors are not able to review the surgeries timely.

### SUMMARY

It is therefore object of the present invention to provide an objective, quantitative, quick, and reproducible feedback based on a surgery video as well as to provide a tool for training.

This object is solved by a method for giving feedback on a surgery and a corresponding feedback system as disclosed herein.

The method for giving feedback on a surgery may be used for training surgeons regarding different kinds of surgeries, in particular eye surgeries such as cataract surgeries, corneal refractive surgeries, glaucoma surgeries, or retina surgeries, or any other kind of surgeries, such as neuro surgeries, ear nose throat (ENT) surgeries, dental surgeries, spine surgeries, plastic and reconstructive (P&R) surgeries, etc. For this purpose, the feedback method comprises the step of loading/and or receiving video data from one or more surgeries. The video data may originate from any device used during a surgery, for example from operation microscopes, endoscopes, an externally setup camera, an additionally camera attached to an operational microscope mount, or the like, both having single view or stereo view, or may be loaded from a database or storage unit. Such a storage unit may be implemented as any kind of storage unit such as a cloud storage unit or a local data base.

The video data may be provided in any kind of video file format, for example MPEG, AMV, AVI or any other available and suitable video file format. The video data can for example comprise a video, multiple videos, e.g., two videos from each of optical paths of a stereo operation microscope, raw video data or with overlays embedded (e.g., from phacoemulsification machine), with additional meta-data (e.g., patient data from patient records or DICOM attributes) and so on. In any case, the video data comprises multiple still images, i.e., frames, from a surgery. Each frame may show an image of a body part the surgeon is operating on and, optionally, may further comprise any kind of operating tool used by the surgeon. Besides the frames from the surgery, the video data might also include frames of non-surgical activity, such as background of the operation room and/or frames, which show pictures of the respective patient before or after the surgery.

After loading the video data, the video data may be analyzed. Analyzing in this context may refer to any kind of processing of the video data which is suitable to provide information about the video data, for example about the content. In a next step, this information can be used for evaluating the analyzed video data, for example for providing any information to a user regarding an assessment of the corresponding surgery.

During analysis of the video data, the video data may be processed, resulting in analyzed video data. As also described later, the analyzed video data may be for example video data being temporally or spatially segmented or being examined regarding the content or additional information like meta-data. In a next step, the analyzed video data may be evaluated, for example for deriving any kind of assessment such as a score of the video data, as will be described later.

After the evaluation, the result may be output, for example displayed on any kind of display device or unit, or on an end device, such as a tablet, the surgeon is working with. Further, the evaluation result may be integrated into a report, for example into text, and may optionally be printed.

It should be noted that the analyzing and evaluation step may be carried out on any kind of processing device, for example on a local computer (e.g., a clinical computer) or in a cloud computing service, whereas the displaying/outputting step may run for example at the surgeon's end device. Thus, the different steps may be physically decoupled and/or decoupled in time from each other. In another embodiment, all steps may be carried out on the same device and/or may be performed simultaneously. Preferably, the analysis and/or evaluation may take place after the surgery during which the video data has been recorded or captured. Thus, it is preferred that the analysis and/or evaluation are not performed in real-time during a surgery, but at some point in time after a surgery.

Based on the above-described method, it is possible to provide a machine-based, human-independent analysis and evaluation of surgery video data. In particular, the evaluation result may be used as training feedback for a surgeon by giving information about the performed surgery the video data originates from, as will be described later. As the analysis and evaluation is human-independent, the corresponding steps may be performed fast and objective. The method further provides a reproducible outcome as the subjectivity is removed due to the machine-based analysis and evaluation, without the need of human experts being involved.

As described above, the video data may include at least one video file having multiple frames. The video data may originate preferably from one surgery but may also include video data from more than one surgery. In the latter case, when analyzing the video data, the analysis may either automatically be focused on only one surgery or may apply to all contained surgeries. In addition, the video data may comprise meta-data, such as pre-surgery or post-surgery data, patient data and/or recorded data from medical devices. This additional information may be included in the video data as meta-data, as overlaying information or as additional files being provided together with the video file.

The video data may be uploaded manually by a user or may be automatically uploaded by a medical device from which the video data originates or from a local data collecting system. In the latter case, the local data collecting system may handle uploading of the video data. The video data may be automatically analyzed and/or evaluated after the video data has been uploaded and/or stored, or when uploading and/or storing the video data has started and there is already enough data available to start the analysis. Further, analyzing and/or evaluating the video data may be started on demand, for example based on a user input. When video data is uploaded, the video data may be assigned to an individual user and the evaluation results may also be assigned to the individual user.

According to an embodiment, analyzing the video data and/or evaluating the analyzed video data is carried out at least partially using a machine learning algorithm. Machine learning algorithms may provide a powerful technical solution for analyzing the video data and/or evaluating the analyzed video data without the need of human interaction. Such algorithms provide the advantage that, once trained, they can be applied with minimal costs and may perform the described method fast and objective. For example, video data, analysis results and/or evaluation results from previous surgeries may be used as training data sets. Further, machine learning algorithms, which may be also referred to as self-learning algorithms, may be implemented for example using neural networks. Further, they can be trained, or fine-tuned, continuously during the analysis and/or evaluation of video data. Although some specific examples will be mentioned in the following description, it should be noted that any kind of machine learning algorithm or machine learning model may be used as will be acknowledged by a person skilled in the art. Also, any kind of machine learning algorithm which will be developed in the future may be used as long as it is suitable to provide an analysis and/or evaluation of video data as described herein. Further, it should be noted that also different machine learning algorithms may be used for different steps of the herein described method when suitable.

According to a further embodiment, analyzing the video includes at least temporal semantic video segmentation (which may be carried out frame-wise), spatial semantic video segmentation (which may be carried out pixel-wise), object detection, object tracking and/or anomaly detection. These different analysis methods may be used separately, parallel with each other, or in combination. Further, all analysis methods or only some of them may be used.

All of these analysis methods may be implemented using machine learning algorithms as described above. The information, which are gathered by semantic frame segmentation, anomaly detection and/or object detection and tracking, may be referred to as meta-representations or analyzed video data.

When analyzing the video data, multiple frames of the video data may be segmented, in particular into frames which may be associated with a surgery phase (i.e., frame-wise video segmentation, also referred to as phase segmentation). Such a segmentation may comprise a spatial or temporal classification of video frames or a spatio-temporal classification (also referred to as a video action segmentation). Depending on the segmentation to be performed, different machine learning algorithms may be used. For example, a 2D convolutional neural network (CNN) may be used for a spatial or temporal classification of video frames or a 3D convolutional neural network may be used for a spatio-temporal classification. The convolutional neural networks may include feature learning and temporal learning modules. For example, an end-to-end model may be implemented for video action segmentation composed of spatial and temporal modules trained jointly. As an example for a convolutional network used for recognition of surgical videos, it may be referred to Jin, Y., Dou, Q., Chen, H., Yu, L., Qin, J., Fu, C. W., & Heng, P. A. (2017). SV-RCNet: Workflow recognition from surgical videos using recurrent convolutional network. IEEE transactions on medical imaging, 37 (5), 1114-1126.

Machine learning algorithms may also be used for determining the current or most likely surgery phase for each and every frame. In another embodiment, machine learning algorithms may be constructed to derive for every frame a probability distribution over possible surgery phases.

Common image and/or video recognition technologies consist of encoding and actual recognition. Traditionally, both steps have been solved independently. E.g., in a first step, image representations are engineered which preserve information relevant for the task at hand while discarding remaining data contained in the original data source. This may involve computation of representations based on color, edges, corners, or combinations and derivations thereof. In a second step, a recognition technology from classical machine learning algorithms is applied to the extracted feature representations, e.g., to predict a semantic category for the feature vector. Such classical machine learning algorithms may be Support Vector Machines, Gaussian Process Models, Nearest Neighbor Classifiers, and the like. While this two-step approach of first encoding and second recognition may offer the advantage of encoding prior knowledge over relevant information and/or expected class distributions, it involves much effort, suitable placed assumptions, etc.

With the availability of deep learning, encoding and recognition may be trained jointly, by optimizing at least one joint optimization criterion, e.g., recognition accuracy. This may also be referred to as end-to-end-learning, and may be realized by combining operations, which can be parameterized, into single computation instruction sets, so called deep learning networks. Deep learning networks may comprise convolutional layers to allow learning of translationally invariant features, such as regular convolutions, convolutions separable in space and/or depth, dilated convolutions, graph convolutions, or the like.

Deep learning networks may comprise non-linear activations to allow learning of non-linear relation, e.g., via Rectified Linear Unit (RELU), parameterized RELU (pRELU), exponential linear unit (ELU), scaled ELU (SELU), Tangens hyperbolicus (tanh), Sigmoid, etc. Deep learning networks may comprise normalization layers to reduce impact of signal variations and/or to numerically ease training with computed gradients, e.g., via BatchNormalization, InstanceNormalization, GroupNormalization, etc. The spatial resolution may be reduced during the processing via pooling, e.g., mean-pooling, max-pooling, min-pooling. Processing results from several processing stages (or blocks) may be combined via skip connections, concatenation, summation, etc. Transformer blocks or other kind of attention operations may be used to capture long-range dependencies, e.g., via self-attention layers and/or multi-headed self-attention layers.

Prediction of class probabilities per frame may be carried out with a fully connected layer, e.g., followed by a soft-max activation and an optional argmax operation to predict a single class with largest probability.

In one embodiment, the prediction of phases for a given frame can be done independently of the previous frames or at least independent of the majority of the previous frames. As an example, the probability of phases can be predicted for single frames, e.g., using common classification architectures which can predict the presence of a phase for a given frame. Also a small number of consecutive frames may be combined and treated as hyperspectral image, e.g., by stacking frames. As an example, 64 consecutive frames could be stacked.

In a further embodiment, a first model may be applied to analyze 2D frames independently, and a second model may be applied to combine the 2D results or 2D embeddings into a time-consistent analysis result. Neural networks for modelling temporal relations may for example consist of long-short-term memory cells (LSTMs), may be recurrent neural networks (RNNs), transformers and/or may consist of temporal convolutions. A further embodiment may also consist of one or more models for extracting short and long-term spatio-temporal features, e.g., a 3D CNN, followed by one or more temporal learning models.

In a further embodiment, the temporal relationships can be learned by exploiting neural networks which directly operate on 3D data, e.g., via spatio-temporal deep networks such as 3D CNNs, spatio-temporal LSTMs, spatio-temporal RNNs, etc., and which can therefore learn to capture relations in space and in time.

Training of machine learning algorithms may in general be based on iterative optimization schemes, e.g., optimization schemes which exploit first-order gradients. Such optimization schemes may be parameterized with hyperparameters, e.g., mini-batch sizes, the learning rate, learning rate scheduling schemes, decay factors, etc. Especially for videos, also the length of sub-video clips may be an important hyperparameter. Furthermore, also the deep learning model(s) may comprise hyperparameters, e.g., number of layers, number of convolution filters in a convolutional layer, etc., which also may be adjusted for a specific training dataset. Especially when adjusting several hyperparameters jointly, it was found that an automated selection of the hyperparameter values is beneficial, which is also referred to as AutoML.

Model parameters are optimized with respect to a loss function or merit function. Such loss functions represent how much a discrepancy between prediction and ground truth annotation shall be penalized in order to change the model parameter values accordingly. For the task of predicting a surgery phase for each frame using a frame-wise temporal semantic video segmentation, a loss function may be one of frame-wise cross-entropy, dice score, truncated mean squared error, F1 score, edit score, overlap score, etc. For a pixel-wise semantic segmentation, a loss function may be any of pixel-wise cross-entropy, pixel-wise overall recognition rate, pixel-wise average recognition rate, intersection over union (IoU), mean IoU, Jaccard loss, etc. For object and/or organ detection, a loss function may be any of precision, recall, intersection over union (IoU), mean IoU, L1 or L2 errors on object parameters, focal loss, dice loss, etc. Further loss functions may comprise a triplet loss, a contrastive loss, a confidence loss, a consistency loss, and/or a reconstruction loss.

A loss function may be configured to reduce the impact of sub-video parts or of frame parts which are annotated with a given class. As an example, a video from the training dataset may contain a sub-video which shows a very uncommon surgery technique which may not be used for training, e.g., when a model shall be trained which shall only recognize standard cataract procedures. In this case, the frames which are annotated to show the specific surgery technique may be masked-out during training, e.g., by reducing their impact during the loss computation or by completely removing the frames from the training frames to consider and/or by skipping the frames during the training process.

For a collected training dataset, the training loss may not be minimizable to zero for a specific machine learning algorithm during the training process of the algorithm. In such cases, it may be beneficial to define at least one stopping criterion at which the training will be interrupted even if the loss did not reach a desired optimal value. Such a stopping criterion can be a predefined number of training iterations and/or a predefined training time and/or a differently predefined training budget, e.g., defined as compute costs in a cloud compute environment or defined as consumed Watt. In a further embodiment, such a stopping criterion may be defined as a specified pattern of the loss evolution over the training time, e.g., as a specified number of training iterations in which the loss values did not decrease or did increase or plateaued or the like (known as early stopping). Such a loss value monitoring may be conducted on a separate validation dataset as to monitor overfitting (i.e., consistently decreasing loss values on the training dataset while observing increasing loss values on a different dataset which may indicate overfitting).

Especially with iterative training schemes as described before, it may be beneficial to iterate several times through the training dataset during the course of algorithm training. One such iteration is referred to as epoch. In each epoch, it may be beneficial to present the training algorithm the examples from the dataset in a different order, e.g., in a randomly permuted order, to reduce unintended side-effects from poorly ordered training datasets.

To increase the generalization ability of a trained algorithm, the training data may be randomly changed during the training (augmentation). In the context of surgery video analysis, such changes may reflect variations in the recording conditions, e.g., changes of lighting, changes of contrast, changes of focus, changes of color tone, changes of overlays including position, color, size, text, and/or borders, etc. Especially for training from videos, such an augmentation may be applied on each frame independently, on sub-videos, or on entire videos.

In a further embodiment, it may be beneficial to select a sub-set of the training dataset for training, e.g., by exploiting active learning techniques and/or by selecting more frames or sub-videos from time points correlated with phase switches or from time points correlated with rare phases or from time points correlated with rare events and less frames of sub-videos from timepoints correlated with standard phases, etc. In a further embodiment, it may be beneficial to adapt this selection of the course of the training, e.g., by selecting in every epoch a different set of frames or sub-videos, e.g., based on the machine learning algorithm classification accuracy at such an epoch, e.g., by selecting the frames or sub-videos which have largest classification errors.

The training of the machine learning algorithm may during the course of the training benefit from exploiting previously trained machine learning algorithms and/or exploiting previously collected training datasets, e.g., by taking previously estimated algorithm parameters as an parameter initialization (also referred to as fine-tuning) and/or by applying domain adaptation techniques and/or transfer learning techniques, e.g., to improve the generalization ability of a machine learning algorithm.

The machine learning algorithms may run on standard processing units, such as CPUs, or on highly parallelized processing units such as GPUs or TPUs. Moreover, multiple processing units may be used in parallel, e.g., via distributed gradient techniques and/or via distributed mini-batches. Furthermore, the operations built into the training scheme and/or the machine learning algorithm may be adapted to match the available target hardware specifications, e.g., by specifying to quantize all operations, e.g., to uint8 or to uint16, instead of operating in the standard numerical regime of full precision (float32) or double precision (float64).

For training machine learning algorithms, training data needs to be collected and annotated. Collection in this context may refer to making data available for the training process, e.g., saving several surgery videos in one storage location. Annotation in this context may refer to associating ground truth information with the surgery videos, e.g., associating every frame of a surgery video with the corresponding surgery phase during the surgery activity. As an example, every frame of a cataract surgery video may be annotated with any of the phase names: idle; incision; ophthalmic viscosurgical device (OVD) injection; capsulorhexis (in the following also referred to as rhexis or continuous curvilinear capsulorhexis (CCC)); hydrodissection; phacoemulsification; irrigation/aspiration; intraocular lens (IOL) implantation; closing/hydrating the wound; non-surgery. As a further example, some of the previous phase names may be split semantically, e.g., frames associated with incision may be annotated as main incision and side ports or limbal relaxing incision (LRI), frames associated with OVD injection may annotated as intraocular OVD injection or external OVD application, Trypan blue application may be separated from OVD/BSS application, frames associated with phacoemulsification may be annotated as chop or nucleus removal, frames associated with Irrigation/aspiration may be annotated as irrigation/aspiration/I/A tip), OVD removal, capsule polishing (I/A tip), irrigation/aspiration (bimanual), or capsule polishing (bimanual), or frames associated with IOL implantation may be annotated as IOL preparation, IOL injection, or toric IOL alignment, also CTR (capsular tension ring) implantation may be distinguished, etc.

In a further embodiment, annotation may also refer to associating individual pixels with the corresponding semantic category. Such semantic categories may comprise (without limitation) body tissue, e.g., iris, cornea, limbus, sclera, eyelid, eye lash, skin, capsule crystalline lens etc., operating tool, cannulas, knives, scissors, forceps, handpiece/tool handles and cystotome, phaco handpiece, lens injector, irrigation and aspiration handpiece, water sprayer, micromanipulator, suture needle, needle holder, vitrectomy handpiece, Mendez ring, biomarker and other markers, etc., blood, surgeon hands, patient facial skin, operational anomalies, e.g. tear, skin or anatomical surface scratches or other anomalies, etc., etc.

Such annotations may be provided by persons skilled in the application fields, e.g., medical doctors, trained medical staff, etc. For the annotation of surgical phases, users may specify start point and end point of every surgery phase in the video, and every frame within this interval may than be understood as belonging to this surgery phase. Such annotation input may be obtained by entering numerical values, e.g., into data sheets or tabular views, or by receiving input from a graphical user interface which may visualize the video and the associated annotations, and which may allow a user to adapt the annotations, e.g., by dragging start and/or end point visualizations or the like. An interactive annotation procedure may be used to reduce the annotation efforts by assisting in creating annotations or proposals, e.g., by exploiting a previously trained phase segmentation algorithm and present the predictions on a graphical user interface for refinement to the user.

Especially for medical applications, it may be beneficial to obtain annotations from multiple persons for the same video data or even to obtain multiple annotations from the same person for the same video data in order to assess annotation variability among annotators (so-called intra-annotator agreement) and repeatability on individual annotators (so-called inter-annotator agreement). Both aspects may be used in various ways during the training part of the machine learning algorithm. As an example, only those videos or sub-videos may be used for training for which the majority of the annotators give consistent annotations. As a different example, it may be beneficial to exclude annotations from individual annotators from the training set if a significant discrepancy between this annotator and other annotators is observed. As yet another example, the annotation variability may be interpreted as an annotation certainty or annotation uncertainty, and this uncertainty may be respected during training when updating the parameter values of the machine learning algorithm, e.g., by adjusting the impact of errors in video regions with high annotation uncertainty to contribute less to the parameter updates than video regions with low annotation uncertainty. In yet another example, the annotation uncertainty may be used to adapt the annotation values, e.g., without considering annotation uncertainty, a phase segmentation annotation may be encoded as a so-called one-hot vector per frame, i.e., as a vector with as many entries as known classes and having a 1 for the index of the annotated class and 0 for all other classes, whereas, with considering annotation uncertainty, the entries may be set different from 1 and 0 and may correspond to distribution of annotations obtained from the annotators, e.g., if two annotators specified frame t belonging to phase 2 and a third annotator specified frame t belonging to phase 3 then the annotation vector for frame t may have two-third on index 2 and one third on index 3.

In some embodiments, it may be beneficial to apply more than one machine learning algorithm during the analysis. As an example, it may be beneficial to apply one phase segmentation algorithm which analyzes the video data at a reduced frame rate (coarse prediction) and apply a second phase segmentation algorithm which analyzes the video data at the original frame rate especially on the frames being in close temporal neighborhood to transitions between phase segments as predicted by the first frame classification model. Such a combination of predictions from multiple models may lead to a reduced computation effort, because the processing unit is only instructed to process the computation-intensive second phase segmentation algorithm on a subset of the original video data. Also other combinations of multiple machine learning algorithms are possible, e.g., as an ensemble.

After analyzing the video data with a first machine learning algorithm, the obtained analysis result might be of insufficient quality, e.g., due to limited training data, due to numerical rounding issues, or the like. It may be beneficial to apply at least one additional analysis algorithm, e.g., as a post-processing step.

Such a post-processing may be based on prior assumptions on the application case, e.g., on plausible and/or implausible transitions between surgery phases which may be given by application experts or derived from training data. As an example, the sequence of phase predictions for each frame of a video can be processed with a Viterbi algorithm to obtain the a-posteriori most probable sequence of phases given the initially predicted phase probabilities per frame as well as given probability values for the first phases and transition probabilities which may have been estimated from annotated training data during training. Alternative processing solutions may be similarly beneficial, e.g., based on conditional random fields (CRFs).

In a further embodiment, predicted phases which do not exceed a specified minimum length may be considered as incorrect prediction and may be associated with an adjacent phase segment. As an example, a predicted phase of less than X second duration (e.g., 1.5 second duration) may be assigned with the predicted phase type of the previous phase, i.e., the phase which ends directly before the short phase starts. In another embodiment, the predicted phase probabilities per frame may be smoothened over a predefined number of frames before concluding the predicted phase type per frame (smoothening). In yet another embodiment, the predicted phase type per frame may be smoothened by a voting scheme, e.g., based on majority voting, within windows of a predefined number of frames, and/or based on predictions from an ensemble of models, and/or based on estimated uncertainty values.

It may be beneficial to incorporate explicit rules which reflect knowledge and/or assumptions about the application case which thereby extends the information which is present in the video data. As an example, during a cataract surgery, short periods of time may occur in which no surgery-related activity is visible in the raw video data, hence, the raw video data may not reveal sufficient information to analyze these periods of time correctly. In consequence, a machine learning solution which only has access to the raw video data might recognize these short periods as idle activity, which may later be accounted for KPIs that focus on idle time and/or overall surgery efficiency. In these cases, however, the surgeon may eventually not be idle, but he/she may switch surgery tools, i.e., put aside tools used in the previous surgery phase and pick up tools necessary for the next surgery phase, but this may happen outside of the field of view of the recording device. Hence, for such short periods of time, it may be advantageous to replace the predictions from the machine learning algorithm by assigning the short period to the previous phase, or to the following phase, or by assigning it in parts to the previous and the following phase.

As mentioned earlier, the raw video data may also include frames of non-surgical activity, such as background of the operation room and/or frames, which show pictures of the respective patient before or after the surgery. A machine learning algorithm may have been trained to recognize such frames and to assign it to a separate phase, e.g., non-surgery activity. In a further embodiment, a machine learning algorithm may have only be trained on video data showing surgery activity. In this case, phase predictions on non-surgery activity may be unreliable. A separate processing solution may be beneficial in such scenarios, which can distinguish frames from surgery activity and non-surgery activity and/or which can recognize start point and/or end point of a surgical activity in video data. With such a solution, the initial phase predictions may be overwritten in the detected non-surgery part or in the parts before the start point and/or after the end point and changed to a predefined value, e.g., a specific phase corresponding to non-surgery activity. In a further embodiment, the entries in the prediction of the first machine learning algorithm which correspond to the non-surgery parts may also be dropped. Such a separate processing solution may be realized as a separate machine learning algorithm, e.g., as a binary classification deep neural network. It may also be realized as an additional part of the first machine learning algorithm, e.g., as a multi-task model which predicts jointly phases and surgery activity. Further, it may also be realized by classical image processing, e.g., by analyzing distributions on gradients, color distributions, and the like, and determine start and/or end points of surgery activity based on changes in these distributions over time.

For an intuitive understanding about the surgery, it may be beneficial to visualize the analysis result in the form of a transition graph and to display and/or output and/or print this transition graph in a user-friendly manner. As an example, such a transition graph may have nodes which represent the surgery phases which the previously described algorithms have been trained for to recognize, and the graph may have edges between two nodes with thickness proportional to the frequency of analyzed transitions between these two phases in the analyzed surgery video data.

Even by exploiting previously described machine learning algorithms and powerful post-processing solutions, users may upload video data not related to the training data, e.g., users may upload video data from very difficult surgery procedures and/or with new surgery techniques and/or with surgery tools that have been unknown or not captured at the time of algorithm training. In such cases, the analysis results may be unreliable and may even be wrong. Furthermore, users may upload video data which is not following the intended use, e.g., users may upload video data from sport events, which may also lead to unreliable and/or wrong analysis results. In such cases, it may be beneficial to analyze the analysis result by technical means, e.g., by counting how many phases of a specific phase type, e.g., how many rhexis phases, have been recognized in the video data. If the number of phases of a specific phase type exceeds a predefined number based on the application case, e.g., if more than three rhexis phases have been recognized in one surgery video, than the analysis result may be understood as unreliable and a remark may be associated with the analysis result or may be outputted or displayed or may be used in a different way to notify the user. Such a solution may analyze the analysis result for a required minimum number of phases per phase type and/or for a required maximum number of phases per phase type and/or for required minimum and/or maximum durations of phases of a phase type and/or for overall number of recognized phases during an entire surgery, or the like. If such a solution analyzed the analysis result as unreliable, then the user might be asked for verification and/or for correction of the analysis result, as will be described later in more detail. Alternatively, the analysis result may be associated with a remark expressing the suggestion for a verification and/or correction by other persons than the user.

The analysis of the video data may further comprise a spatial semantic video segmentation. This semantic segmentation may be carried out pixel by pixel (spatial) with common segmentation architectures, e.g., based on encoder-decoder architectures or vision transformer architectures. In a further embodiment, the segmentation can also be applied jointly on temporally correlated frames, e.g., on the entire video as is or on sub-videos, e.g., by applying transformer encoder-decoder architectures on sequences of frames, or by using skip-convolutions, or by using spatio-temporal semantic segmentation architectures common segmentation architectures with 3D operations such as 3D convolution, 3D pooling, etc.

In such embodiments, every pixel of a frame may be assigned to a semantic category or to a probability vector representing the estimated probabilities of this pixel belonging to several or all possible semantic categories. In a further embodiment, every pixel of a frame may be assigned to a unique instance of a semantic category (instance segmentation) which may be beneficial to distinguish multiple tools from the same tool type present at the same time. Such semantic categories may comprise (without limitation) body tissue, operating tool, blood, surgeon hands etc. as described and listed above.

A post-processing step may consist of energy-minimization-based techniques, e.g., for spatial and/or temporal smoothing of predictions in local neighborhoods, or for improving alignment of edges in predictions and video data, e.g., by applying conditional random fields (CRFs) with pairwise or higher-order potentials.

In addition, or alternatively to a semantic video segmentation, the analysis of the video data may comprise an anomaly detection. In this case, it may be determined for every frame if an unknown event is happening. An unknown event or anomaly in this context may refer to an event which is not expected to happen. Such an unknown event may be identified for example with respect to a previously collected training dataset, which may comprise for example only “standard” surgeries without any anomalies. The trained datasets may correspond to analyzed video data which has been verified, as will be described below. However, it is also possible to identify the type of anomaly by using a training dataset, which includes different types of anomalies. Using this approach, it is possible to identify not only the general presence of anomalies but also the concrete type of anomaly.

A detection of anomalies may be with respect to sub-videos or individual frames or even with respect to individual pixels or pixel sets. For anomaly detection on pixel levels, reconstruction-based algorithms may be exploited. For example, a machine learning algorithm may be trained to reconstruct frames from a given dataset such that the reconstruction error is minimized under the constrained of limited model complexity. Thereby, the algorithm may learn a suitable representation of the training data. Such a solution may be built with principal component analysis (PCA), Auto-Encoders, Deep Auto-Encoder, Deep Variational Auto-Encoders, Deep Hierarchical Variational Auto-Encoders, auto-regressive generative deep neural networks, or the like. Presented with frames of previously unseen videos, such a reconstruction algorithm may result in low reconstruction errors for frames which are visually similar to the training dataset, and to larger reconstruction errors for frames with anomalies. As an example, regions showing strong blood flow may not have been captured in videos of the training dataset and may hence not be reconstructable with small errors. By comparing reconstruction and frame, a reconstruction difference can be derived, which indicates anomalies in regions that show larger difference than regions which show low differences. Such a reconstruction analysis may be even more reliable by sampling several reconstructions from a reconstruction algorithm and deriving an anomaly map not only based on a single reconstruction but also by taking variations in the multiple reconstructions into account.

In a further embodiment, the detection of anomaly may happen on a frame level or sub-video level, which may also be referred to as abnormality detection or out-of-distribution (OOD) detection or novelty detection or one-class classification. In such an embodiment, it may be beneficial to represent frames of sub-videos with feature vectors, and to learn novelty detection algorithms from feature vectors derived from frames or sub-videos of the training videos. Such novelty detection may be parzen density estimation, Gaussian mixture models (GMM), support vector data description (SVDD), one-class support vector machines (1-svm), Kern-Null-Foley-Sammon-Transformation (KNFST), Gaussian Process regression models, or the like. In a further embodiment, an anomaly estimation may be derived by uncertainty estimation methods, e.g., by treating predictions with larger uncertainty as more likely to be anomalous than predictions with low uncertainty. In such embodiments, uncertainty estimation may be realized by computing epistemic uncertainty from model ensembles, Monte-Carlo techniques such as Monte-Carlo dropout, etc.

In a further embodiment, the entire video may be inspected for being anomalous, e.g., with respect to the training dataset. Such an anomaly detection of video level may be realized by deriving statistics from the prediction of machine learning algorithms used for video analysis, e.g., by deriving statistics about number of recognized phases, duration of phases, occurrence information for detected tools, etc., and to compare these statistics with statistics derived from videos in the training dataset and/or with expected values or intervals for such statistics. Such a comparison may be realized by computing differences between the derived statistics from the video and at least statistics of at least one video from the training dataset and comparing these differences against pre-defined tolerances. In a further embodiment, such a comparison may be realized by an anomaly detection model trained on the statistics of at least one video from the training dataset and using the resulting anomaly score computed by such a trained anomaly detection model for the new video as indicator for anomaly of the entire video.

Furthermore, the analysis of the video data may comprise object detection and/or tracking within the video data. Thereby, individual tools may be localized in every frame and/or their trajectory may be computed over time. Such an object detection may be realized by regressing parameters which describe the location of a tool in a given frame, e.g., by regressing parameters of an enclosing bounding box. Such a parameter regression may be realized by anchor-based methods, e.g., by regressing bounding box parameters relative to specified locations. In a further embodiment, such a parameter regression may be realized by anchor-free methods, e.g., by fully convolutional networks for key-point regression. In a further embodiment, an object detection may be realized by a two-step approach, which may consist of a location proposal algorithm and a proposal classification algorithm.

The localization of the tools, i.e., the object detection, may be combined with the semantic temporal frame segmentation. Since specific tools are only present during certain parts of a surgery, this might be helpful to distinguish between the different phases.

For tracking objects over frames, tracking by detection may be applied which may associate closest detections in two given frames as track, e.g., by analyzing motion, shape, appearance, or similar to derive the correlation between detections.

In a further embodiment, an end-to-end approach may be realized for simultaneous detection and tracking, e.g., an encoder may be used, e.g., a classification model, for spatial localization and scene understanding to create a stationary graph of objects of interest within a scene, and a temporal decoder may be used to take the output of the spatial encoder as input in order to capture the temporal dependencies between frames and infer the dynamic relationships by creating a dynamic scene graph.

As mentioned earlier, in some embodiments, it may be advantageous to construct a machine learning algorithm which jointly performs at least two of the previously described techniques. As an example, a deep neural network may be constructed which predicts for every frame the surgery phase and simultaneously predicts per pixel of the frame its semantic category. This may also be referred to as multi-task learning.

Analyzing and/or evaluating the video data may be improved by including additional pre-surgery or post-surgery data as mentioned above. For example, such additional patient data from patient records (e.g., pre-operation data) may be a measured white-to-white distance which may be used for a reliable evaluation of a rhexis roundness. Additional data may further include information from medical devices like the tracked location of the iris, additional recordings from other devices in the operating room like phacoemulsification energy recordings to derive gentleness of phacoemulsification usage.

According to a further embodiment, evaluating the analyzed video data includes detecting at least one event of interest within the video data and deriving at least one score from the at least one event of interest.

Events of interest may be any feature or characteristic of the surgery which can be used for indicating important processes within the surgery. For example, an event of interest may be a specific surgery phase. Further examples of an event of interest or score are an idle phase, an optical focus during the surgery (e.g., a medical device being focused or being out of focus), a presence of a tool, information about a surgery infrastructure (e.g., illumination etc.), any other feature of the surgery and/or any tool used during the surgery. The events of interest can be detected based on the analyzed video data which has undergone phase segmentation, pixel-wise semantic segmentation, object detection and tracking, and/or anomaly detection and so on.

For each detected event of interest, one or more scores may be derived. When multiple events are detected during the video, which correspond to the same process within the surgery, for example incisions, they may be associated with the same score, e.g., incision attempts.

The derived scores may then be used to enable a user, for example the surgeon who has conducted the surgery associated with the video data, to get a feedback regarding the conducted surgery. Such a score may also be referred to as a (medical) key performance indicator (KPI) or clinically relevant metric. A KPI or score may be derived directly from the analyzed video data, for example as a specific characteristic of the surgery, e.g., incision attempts or the like.

In order to get an assessment regarding the quality of the conducted surgery, evaluating the analyzed video data may further comprise determining a score value for the at least one score. The score value may be determined as an absolute value, for example number of incision attempts or absolute phase length, only based on the currently conducted surgery. This may provide the advantage of a feedback without the need of other comparative data.

Alternatively, at least one score may be compared with stored data, in particular with historical data of other, in particular previous surgeries, wherein determining a score value of the at least one score is based on the comparison result. In this case, the score value may provide an assessment of the currently conducted surgery compared with a reference value, e.g., a predefined, particularly absolute, value or a reference value from other, previous surgeries, for example of a highly skilled surgeon, or any other kind of suitable reference value.

In particular, the derived scores may be compared with stored scores of other surgeries, resulting in score values. Preferably objective, for example numerical, score values, may allow an easy comparison to other surgeries, for example of skilled surgeons or experts. Thus, the at least one score and the corresponding score value may be used to compare the current surgery with other (preceding) surgeries. This may be done for example using detected objects, tracked objects, detected anomalies etc. as described above.

For example, when evaluating the analyzed video data, a presence of a tool may be detected as one event of interest. From the presence of the tool, scores may be derived, for example relative and/or absolute position of the tool, speed of the tool, etc. These scores are not quantitative, but define possible parameters of the respective event of interest. In the next step, the scores may be quantified, i.e., the score values for the respective scores may be determined. This may be for example a speed value for the speed of the tool.

In another example, information about a surgery infrastructure may be detected as one event of interest, wherein the information about a surgery infrastructure may be an illumination. The score may be a condition of the illumination and the score value may be 1 (activated) or 0 (deactivated).

Further, a specific surgery phase may be detected as an event of interest. From this event of interest, the scores frequency of the surgery phase or length of the surgery phase may be derived. For the score “length of the surgery phase”, the absolute length of the surgery phase or a relative length compared with the score “length of surgery” from another surgery may be determined as score value.

In some cases, the event of interest may only have one score, in which case the event of interest is at the same time also the score.

In one embodiment, it may be beneficial to derive the scores not from the analyzed data, but to directly derive the scores from the video data, e.g., by using a machine learning algorithm. Thus, analyzing the video data may include in this case directly deriving at least one score value for at least one defined score and/or event of interest. As an example, for the case of determining the roundness of a performed rhexis, the two-step solution may consist of first tracking and/or segmenting the rhexis (i.e., analyze the video data) and then computing the roundness from the segmented rhexis positions (i.e., evaluate the analyzed data), while a one-step solution may consist of a single machine learning algorithm which was trained to predict the rhexis roundness directly from the video data, e.g., as a single scalar.

A user may select the scores regarding which score values are to be determined. Further, a user may also select, after analyzing the video data, which scores are to be computed. This may reduce the needed computational resources and time as the analyzed video data is not evaluated with respect to all possible scores but only to pre-selected scores. Such a selection may also be applied when displaying the evaluation result. This means that only selected scores might be displayed and/or outputted.

When determining a score value, the user or surgeon may select a target group (e.g., surgeons with the same level of expertise, himself from previous surgeries, expert surgeons, advisors from the same clinic, etc.) from which the stored data are chosen. The at least one score may be compared against the data from this target group. For example, the at least one score may be compared with an average score of the target group. In one embodiment, the target group may be pre-selected based on the current user, e.g., based on his last selection, or based on his associated clinic, or based on his score level.

As mentioned above, an event of interest or score may be for example a specific surgery phase. When comparing the length (score) of this surgery phase of the current surgery with the length (score) of a surgery phase of a preceding surgery, a score value may be derived which indicates how good in terms of speed the current surgery, i.e., the surgeon, has been compared with other surgeries or surgeons. Alternatively, the absolute length of the surgery phase may directly be used as score value.

Further score values, corresponding to the above mentioned scores, may be for example, without limitation: maximum length of a surgery phase; minimum length of a surgery phase; consistency of a phase length over a period of time; length of idle phases; focus during the surgery with regard to patient eye; centration of the operational microscope with regard to patient iris during the surgery; number of incision attempts; quality of instrument handling (e.g., estimated tool velocity, lack of unnecessary movements, . . . ); rhexis quality (size of rhexis, circularity of rhexis, number of grabs and releases); number of enters/exits of instruments in the eye; smoothness of instrument movements in the eye (depending on surgery phase); centration of movements in the eye (depending on surgery phase); tilting of the eye when an instrument is inserted during incision and paracentesis; tilting of the tool tip (e.g. not going too deep), analysis of complications (length of complication, phase, . . . ); size, adjustment and consistency of incisions (e.g. with regard bleeding); cleanness of pupil after irrigation/aspiration, capsule polishing; quality of surgical scene preparation (e.g., draping, . . . ), etc.

According to a further embodiment, the method further comprises determining an overall score based on a combination of the derived scores. In case more than one score is derived per video data, an overall score may be determined which gives a summarized feedback regarding the complete surgery. Such an overall score may be identified by summarizing the individually derived scores or as an average value of the individual derived scores. Other implementations may also be possible. The individually derived scores may also be differently weighted so that specific scores have a higher weight than others, for example based on their importance or significance for the particular surgery. Further, a combination of an overall score and individually derived scores may be used or it can be switched between an overall score and individually derived scores, for example based on a user choice.

It should be noted that the steps of analyzing the video data and evaluating the analyzed video data may also be combined or merged. In particular, a score or event of interest may be derived without complete analysis of the video data.

According to a further embodiment, the feedback method further comprises visualizing the evaluation result, and preferably the score value, in particular using bars, pie charts, trend graphs, overlays, or as text etc. The visualization of the evaluation result may be provided to a surgeon who has conducted the surgery. For example, the evaluation result may be visualized on a screen directly connected to a local computer or on a separate device, e.g., a smartphone or tablet which may receive evaluation result via network. In a further embodiment, the visualization of the evaluation result may be provided to a person who has not conducted the surgery, e.g., to a second surgeon or to a supervisor. This can be beneficial for reviewing cases from different surgeons to compare and to learn. The visualized evaluation results may also be outputted into a written report.

According to a further embodiment, the feedback method further comprises filtering the displayed evaluation result according to a user input. For example, the evaluation results which are shown can be filtered to only present to a surgeon the evaluation results he/she is most interested in. The user input may be received via any suitable human-machine interface. The displayed evaluation result may also be filtered using any further suitable filter method.

According to a further embodiment, the feedback method further comprises refining the analysis and/or evaluation based on a user input. This may further improve the accuracy of the analysis and/or evaluation. For example, the user input may verify and/or correct the analyzed video data. Verifying in this context may refer to a confirmation or rejection of the analyzed video data. Correcting in this context may refer to altering or editing the analyzed video data. In one exemplary embodiment, correction of a phase segmentation can be done by adapting the start point or end point of a predicted phase segment.

For correcting the analyzed data, there exist many possible approaches, for example depending on the respective analysis method. Some examples will be given in the following, without being exclusive. When correcting the analyzed video data, the analysis result may be output or displayed to a user so that the user can review the analysis results.

For instance, all detected surgery phases may be displayed on the display unit. The surgery phases may be shown in one timeline or one timeline per surgery phase may be shown. When correcting the surgery phases, a user may select a point within a timeline, e.g., by double-clicking, to specify a surgery phase at this point of time. Start and end points of surgery phases may be adjusted by shifting the current phase boundaries, e.g., using click and drag.

For correcting a phase detection, the correction may refer to merging two or more phases, deleting one or more phases, change a phase type, etc. For correcting the tool detection and/or tracking, bounding boxes (which define pixels within a frame which are considered to relate to a tool) may be adjusted by moving, adjusting size, adjusting corners, adjusting class of box etc. For correcting a segmentation, in particular a pixel-wise segmentation, several graphical methods may be used like brush-based correction, polygon-based correction, etc.

Refining the analysis and/or evaluation may improve the quality while the analysis and/or evaluation may still be performed automated and therefore objective. Further, the transparency for a user may be improved as the user obtains control particularly over the analysis which is otherwise hidden and only the resulting scores are accessible.

An exemplary feedback method may thus load the video data and analyze the video data as described above, e.g., including frame segmentation, tool tracking, etc. Subsequent to the analysis, the user/surgeon, may review the analyzed video data. For example, the user verifies whether a tool was tracked correctly, whether the semantic categories of the frames are correctly identified, whether an anomaly was detected correctly etc. In addition, the user may also correct or refine the analysis if necessary. This may be done via a user interface, like a display unit and a corresponding input device (mouse, keyboard, microphone for speech recognition, etc.), on which the analyzed video data is shown and via which the user may carry out the verification or correction.

In one embodiment, the user can be asked to verify and/or correct the analysis result if it deviates from an expected analysis outcome. Such a deviation check might be understood as a technical safety-net solution. In one embodiment, this deviation check might consist of comparing the predicted occurrences per phase type with a maximum number of expected occurrences. As an example, in a standard cataract procedure, the maximum number of expected incision phases per surgery may be five, and any analysis outcome which predicts more than five incision phases during one surgery might indicate a deviation which should be verified and optionally corrected by a user.

In another embodiment, such a deviation check might consider the analyzed time per recognized surgery phase, and a deviation from minimum and maximum expected phase time can be checked for. As an example, an incision phase in a standard cataract surgery might require at least 3 seconds and might not take more than 300 seconds.

In one embodiment, the user might receive a notification if any of such a deviation check is leading to a deviation and this notification may include the specific type of deviation. In another embodiment, the user might receive only a notification of deviation without further details on the type of deviation. In one embodiment, the user might be directly led to the specific part of the surgery in which the deviation occurred to verify and optionally correct the analysis data without the necessity of verifying the remaining parts of the analysis data.

After the verification and optional correction, the feedback method may continue with the evaluation of the verified and/or corrected analyzed video as described above. This verification and/or correction provides the advantage that the user may interactively correct or refine the analysis of the video data, which in turn improves the evaluation of the analyzed video data. Such a correction step can be especially advantageous in surgery cases which do not follow the expected surgery protocol, e.g., due to unforeseen complications or due to non-standard surgery techniques, and which are therefore not well represented in the training data used for training the at least one machine learning algorithm which performs the analysis and/or evaluation.

According to a further embodiment, the verified analyzed video data may be used for training of a machine learning algorithm for analyzing the video data. This may be implemented in form of a feedback loop so that the machine learning algorithm receives the corrected and/or verified analyzed video data as input training data. This input training data can also be used to extend the previously available input training data. In these scenarios, it might be especially advantageous to retrain the machine learning algorithm by taking a previously trained solution for the machine learning algorithm into account, e.g., by transferring the previously estimated algorithm parameters as initialization to the new training step (also known as transfer learning or fine-tuning). In various embodiments, the training can also be realized as continuous learning and/or as online learning. In one embodiment, it may further be decided for the verified and/or corrected new training data if it shall be used for training, e.g., decided by a human or by a relevance estimation technique as known from active learning scenarios. Using the verified and/or corrected analyzed video data for training may lead to more accurate and/or more robust trained machine learning algorithms, which over time may also reduce the efforts for verifying and correcting the analyzed video data as the machine learning algorithms become better and better in processing of the video data due to reviewed and corrected analyzed data.

Parts of the analyzed data could also be completely removed from the score computation, e.g., if the analysis of the machine learning algorithm is too wrong and the user would not want to correct it. Parts of the analyzed data could also be marked for verification and/or correction by a third person, e.g., if the user is not satisfied with the analysis, but also not want to correct it him/herself, the analyzed part can be flagged as “verify&refine”, which may be later done than by a third person, and only after the verification by the third person, the result, i.e. the analyzed and verified and/or refined video, may be included in the evaluation.

According to a further embodiment, the user input may define the kind of analysis procedure and the method may further comprise analyzing the video data using the defined kind of analysis procedure. According to this, the analysis may be further improved by an additional user input. For example, the user input may indicate desired features, e.g., higher focus on specific phases, preference over smoother phase segmentation, a specific metric, etc.

As mentioned above, the method may further comprise selecting a machine learning model based on the defined kind of analysis. For example, a machine learning model may be selected which is suitable for the desired features according to the user input. The method may select the suitable machine learning model out of a pool of models or may train a model incorporating the requested features. E.g., a user may only care about phacoemulsification and capsulorhexis and may select those as desired phases. As such, a model for classification of only these phases may be picked from an existing pool of machine learning models and used for analysis and/or evaluation. Also, machine learning algorithms or models may be used and selected which are focused on individual interesting aspects, e.g., a machine learning model which only detects incision attempts. If no suitable machine learning models exists, the method may select a machine learning model which is the best suitable one and may train this machine learning model accordingly, for example based on the desired features as input training data.

As described above, the video data may be different kind of video data, for example raw RGB video, but may also include additional imaging technology, e.g., optical coherence tomography (OCT), which is preferably synchronized with the camera stream, either directly for example by having the same frame rate or by internal synchronization. Such additional imaging data may provide for example a better access to depth information. This imaging data can be exploited as second source of information (e.g., to estimate the incision angle more reliably than only using pure video data). When using machine learning algorithms, such different sources of information, i.e., the raw video data and any additional imaging information, may be integrated into the analysis and/or evaluation by running two machine learning algorithms or models independently, and combining their results (so called late fusion), or by using both information sources in one machine learning algorithm (so called middle fusion or early fusion). Additional imaging data may also be exploited as new, independent source of information where new scores can be derived from (e.g., depth of incision, distance incision tool to lens over time, etc.). The video data may comprise hyperspectral imaging data, intraoperative fluorescence imaging data, cataract surgery navigation overlays, intraoperative aberrometry, keratoscope visualization etc.

The evaluation result may be displayed to the user as described above. However, that does not immediately result in a positive learning outcome because it is just one surgery regarding which the user can see the evaluation result. To get not only a feedback regarding a single, for example the current, surgery but to get a feedback on a learning progress, it would be helpful to take into account multiple surgeries over time.

For getting feedback on a surgeon's learning progress, a surgeon currently needs to rely on questionnaires, or on manual score systems, on educational standards and assessments, or on residency programs. All of these options are effort-intensive, as they involve manual consulting, are subjective and not representative. Furthermore, existing solutions are often focused on residency surgeons and do not apply well to experienced surgeons who still want to learn, want to standardize, and want to improve.

Therefore, according to a further embodiment, the method comprises tracking a learning progress of a user and/or a user group based on the evaluation result, in particular of multiple surgeries. Tracking the learning progress may provide the advantage that the user may get a feedback not only to one conducted surgery but to multiple surgeries and his/her development over the multiple surgeries. This may be especially beneficial since a single surgery may face unexpected complications which for example might lead to an overall increased surgery time that might not be representative for a user's performance on standard surgeries. When tracking the learning progress over multiple surgeries, one surgery being abnormal may be compensated by other surgeries without such unexpected complications. Thus, tracking a user's scores over multiple surgeries allows a more comprehensive and representative analysis of his/her learning progress.

Likewise, when tracking a learning progress of a user group, the user group may get a feedback on the surgeries of the selected user group and/or over time. Chain clinics might use this learning tracking for checking whether a standardization goal has been reached, for example by determining whether the learning progresses of their surgeons converge towards standardized practices and workflows. Further, the tracking may be used by clinics for advertising the education of their surgeons or, in the case of an insurance claim, to prove the (continuing) education or training of their surgeons. In particular, the user group which shall be tracked can be selected by the current user or can already be preset to a default, e.g., to all surgeons from the same clinic as the user.

The learning progress may be determined by comparing the evaluation result of one surgery with at least one further surgery of the same user and tracking the learning progress based on a result of the comparison. In particular, the learning progress may be determined by comparing one or more score values of one surgery with the corresponding score values of a preceding surgery. If the score values are getting better, the surgeon is considered to make a learning progress. The determined and achieved learning progress may be displayed by the display unit or may be outputted into a report. In a further embodiment, the achieved learning progress may also be stored, e.g., in a database, on a local hard drive, or in a cloud storage, for future access.

Examples of scores, which may be used for tracking a learning progress may be a duration of the surgery, intraocular lens (IOL) positioning duration, roundness of rhexis shape, deviation of rhexis shape from an intended shape, phacoemulsification efficiency, IOL implantation smoothness, correlation between complication rates, phacoemulsification power, liquid usage, refractive outcome, etc. In general, any numerical score value described earlier which allows for at least ordinal comparison or which can be interpreted as an ordinal or even metric value can be used for tracking a learning progress. Further, categorial score values may be used for tracking a learning progress. In this case, instead of comparing numerical values, categories (for example kind of surgery operations) may be used for comparing with other surgeons, e.g., percentage of one surgery operation within a surgery.

As no additional mentors are required for scoring and tracking the learning progress, no manual bookkeeping of examination scores, etc. is needed. Further, the method is suitable for surgeons in all expertise levels. Since all surgery videos of a user/surgeon may be used for evaluation and tracking of the learning progress, the overall learning progress is stable trackable and will be less influenced by single exceptionally good or bad surgeries. Further, since the scores are tracked without manual involvement, the overall tracking of the learning progress is objective, and it can be updated frequently with every new video of a user.

According to a further embodiment, the scores which are used for tracking the learning progress may be selected manually, for example based on a user input. Thereby, one or more scores may be selected to be tracked. In another embodiment, the scores to track may be pre-selected, e.g., based on the users last selection, based on his specified preferences, based on scores specified by his clinic, based on his associated user group, or the like.

A summary or report on the learning progress, which may either be displayed or outputted for example in text format, may be generated on demand, after every newly uploaded and analyzed video data, if the value of a selected score changes significantly (this may be defined by the user, e.g., more than 5%). In addition to the learning progress, such a report may also comprise specific information about the most recent video, e.g., a summary of the last surgery with regard to conducted phases, time per phase, etc. Furthermore, such a report may additionally contain at least one sub-video of the video data or a combination of more than one sub-video of the video data, wherein such a sub-video may correspond to at least one event of interest which was analyzed in the video data. As an example, a user may select at least one of the recognized surgery phases from the surgery video, and every selected phase will be contained as a sub-video in the report or newly created videos consisting of some of the sub-videos from the selected surgery phases.

As described above, the progress of an individual user may be tracked for personal education purposes. In this case, the tracked progress may be shown only to the user himself, e.g., a surgeon can track his scores on his own uploaded surgeries over time. Optionally, the surgeon may also compare his score against other selected surgeons or surgeon groups. Further, the progress of a surgeon or surgeon group may also be tracked by an employer or supervisor to evaluate the performance and/or training of the respective surgeon.

When tracking the progresses of a user group, the tracked progress may be computed as a whole, over the complete group, and individually, for each user of the group. When displaying the learning progress of such a user group, the top-performing and low-performing surgeons may be highlighted, selected and/or shown based on the selected and tracked scores.

According to a further embodiment, the feedback method further comprises predicting a development of the learning progress based on the tracked learning progress and/or stored learning progresses of other users. Such a prediction provides the advantage of that it may be available over all expertise levels and without areal limitation (i.e., can be available also outside of a clinic etc.), and may be personalized. The predicted development may provide an overview on how the learning progress will evolve over time or over the next surgeries. In one embodiment, the development may be predicted based on the tracked learning progress, i.e., as an estimation based on the learning progress of the past. In another embodiment, the development may be predicted based on a comparison of the tracked learning progress of one user with the learning progresses of other users, i.e., as an estimation based on comparable or similar learning progresses of others. Also, a combination of these embodiments may be used for predicting the learning development.

According to a further embodiment, the prediction of the development of the learning progress may comprise estimating a time until when a specific learning level, in particular a specific score value, will be reached based on the predicted development. The estimated time may be an absolute chronological time or may be a number of surgeries to be performed before the specific learning level will be reached.

Before predicting the development of the learning progress, the feedback method may further comprise receiving a user input defining the specific learning level. The specific learning level may be for example a specific score value or may be a value for the overall score. By defining such a learning level, the surgeon may specify for a selected score or multiple selected scores which level he/she wants to reach, for example a time goal (average surgery time of less than 10 min), incision attempt goal (average incision attempts less than 3.7 per surgery) etc.

In addition, or alternatively, the actual score level may be determined based on the actual score (e.g., user reaches a sufficient or targeted roundness of rhexis in 80% of surgeries) and then the time may be calculated, based on the predicted future learning progress, when the surgeon will reach his/her specified learning goals. For example, it may be predicted that the user/surgeon will reach the selected score level in 2 months from now, given the current surgery frequency and the current surgery predicted development. Further, it may be predicted how many surgeries might be needed to reach a selected score level. Further specific examples will be given in the following.

In another example, based on the actual development, a trend may be determined, and it may be determined when the user will reach a desired level. For this, scores of the user from previous surgery are considered and extrapolated, i.e., trend analysis is performed. A trend curve determined based on the extrapolation shows when this trend curve will reach a specified target score level.

In another example, a progress prediction may be made via relation to a most-similar surgeon. It may be searched in a data base for the most-similar surgeon who already reached the desired target score level. Such a search could be done based on a most-similar learning progress up to the current score or based on meta-information (age, based on clinics, based on same mentor, based on number of surgeries, based on surgeries of the same type, etc.). To visualize the progress prediction, the current learning progress until the current score level may be shown, overlaid with the learning progress of the most similar surgeon from the current score until the selected target score.

In another example, a progress prediction may be made via the most-probable learning progress based on machine algorithms trained on learning progresses of other surgeons in a database. This prediction is based on the score development of the user, and the learning experience of other surgeons. For this purpose, a time-forecasting machine learning model (e.g., a recurrent neural network (RNN), Gaussian copula model, etc.) may be used to extract the learning progress from all surgeons in the database. The trained model may be applied to the current learning progress and may predict how the score might evolve and when a specified score level might be reached.

Another example refers to the prediction of a probable trend progression and an uncertainty estimation. In this case, not only the progress is predicted, but also a confidence interval. For example, it may be determined that, on average, the learning progress will look like that, but taking into account a standard deviation or on average, the user may require 37 more surgeries+/−9 surgeries.

It should be noted that all implementation examples of predicting a learning progress development are only examples and further implementations are possible. Further, the examples may also be combined.

As described above, video data may be analyzed and evaluated. The results of the analysis and evaluation may be stored together with the video data in a storage unit or database as explained above. This may result in a plurality of videos being stored over time which can be used for learning or training a surgeon. A good learning experience can for example be achieved by visually comparing two videos, e.g., the video of the recent surgery with a reference video. However, such videos need to be selected before comparing them. Also, for comparing the scores from a new video against a reference video, such a reference video needs to be selected first. Finding a suitable reference video in a large and growing video gallery is very complex, which may delay or hinder the learning success.

Therefore, according to a further embodiment, the feedback method may comprise comparing the evaluation result of the video data with evaluation results of one or more other video data and determining a rank of each of the multiple videos based on the comparison result.

It should be noted that the determination of the rank of the videos may be decoupled in time from the analysis and/or evaluation step of the video data. For example, all analyzed videos may be ranked according to a user input at once and/or a newly analyzed video may trigger a ranking of all videos including the new one and/or a user may select to rank all videos after analysis and/or evaluation of the new one is finished. Other possibilities or also combinations of the above mentioned may be implemented.

Such ranking of videos leads to an order of the videos such that related videos are grouped together, based on the evaluation results. Videos having similar or identical evaluation results may be grouped together or at least close to each other. When determining a rank, the rank may be based on a comparison of the evaluation results. Thus, the videos may be ranked according to a score, a score value, an event of interest or the like as described above. Instead of manual ranking or sorting of the videos as in common systems, this may be done automatically, without human intervention.

As the ranking is based on evaluation results which are determined in any case, no additional processing of the video data is necessary. Further, the used ranking based on evaluation results is scalable, as, with increasing number of videos in the gallery, only the computation time of the ranking increases, but not the time of visual inspection or pre-processing of the video data. As the complete video analysis, evaluation and also ranking is machine-based, the ranking will not be affected by human exhaustiveness so that the risk of missing videos with relevant content is reduced. Further, it is repeatable, as the evaluation can be realized in a deterministic fashion such that the ranking does not depend on external factors, e.g., time of search (which would affect a manual search). Moreover, the herein described ranking is not based on meta-data but on the evaluation result of the video data so that no full meta-data record is needed.

In a further embodiment, to provide a user an easy access to the videos, the videos may be displayed in a gallery representation based on the determined rank of the videos. For example, the videos may be sorted in ascending or descending order, based on the comparison result.

As described above, the ranking is based on a comparison of the evaluation result. During such a comparison, a predefined characteristic of the evaluation result may be compared, which may be, for example, be selected by a user.

The predefined characteristic may include a similarity or dissimilarity degree of at least one score of the corresponding video data and/or a difficulty degree of the corresponding surgery.

For example, a user may select one or more scores, the determination of a similarity or dissimilarity should be based on. Thus, the videos in the gallery may be ranked based on a similarity of the available scores of one video to other videos in the database. Examples of such a ranking are: only rank videos based on similar phase length for specified phases, only rank videos based on similar economy of motion, only rank videos based on similar number of incisions. Instead of ranking based on similarity, the videos may also be ranked according to a dissimilarity, e.g., compare a video of a bad phacoemulsification with videos of a good phacoemulsification (in addition, these videos may be similar in other available scores).

When more than one score is selected, the scores may be considered in a specific order, e.g., as desired by the user. For example, it may be selected by the user to rank according to the score “number of incisions” which should have the highest priority in the ranking, and if two videos have the same score value under this score, then the rank may be based on another score such as incision angle.

Alternatively, the ranking may not be tree-based and instead the relative importance per score can be specified. For instance, the similarity of score “number of incisions” may contribute with 60% to the overall ranking, and the similarity of score “incision angle” may contribute with 40%.

It may also be defined, e.g., by the user or as default setting, that only the top-ranked videos may be displayed. This may reduce the overall number of videos being displayed. For example, all videos may be ranked, but only the top X, for example top 10% or the best 15 videos, are displayed, which can be specified by the user.

In a further embodiment, when ranking the videos, only sub-parts of the videos may be considered. The user may specify that only sub-parts of all videos should contribute to the ranking, e.g., restricted to specific phases, restricted to availability of specific tools, etc. It should be noted that a restriction to specific sub-parts may exclude some scores when they are no longer available in the remaining sub-parts or not meaningful anymore. For example, if the ranking is restricted to a phacoemulsification phase, no number of incisions can be computed. In this case, the ranking may skip a selected score if not available and continue with the next available score.

As described above, ranking may also be based on a comparison of a difficulty level of a surgery. For example, the difficulty level or the standardness of a surgery (rate case complexity) may be determined and the videos may be ranked accordingly. For instance, only standard surgeries (i.e., without difficult techniques, without complications, etc.) may be shown. The standardness can be derived by checking performed phases, overall number of phases, number of individual phases, repetitions, phase attempts, number of complications, used tools, etc. A specific machine learning model may be used which is trained to determine the difficulty level of a surgery.

According to a further embodiment, when ranking the videos, also available tutorial videos may be ranked. For example, when a score of the evaluated video, which is ranked and compared with other videos, exceeds a predefined value, a tutorial corresponding to the score may be included in a tutorial video list.

In a further embodiment, the ranking of all videos may be filtered so that there may be multiple ranking results for different video sets. For instance, there may be one ranking for all videos of the same clinic, and a separate ranking for the curated set of “best practice videos”.

According to a further embodiment, the ranking of the videos may not be based on the evaluation result, but on the analyzed data. As an example, the ranking may be determined based on a similarity of recognized phases, and such a similarity may reflect similar order of phases, similar length of phases, similar starting points of phases, etc. In a further embodiment, the ranking may also be based on derived representations of the analysis data. As an example, the ranking may be based on similarity of a transition graph which may be derived from the phase segmentation as already described earlier, and such a graph similarity may reflect similar values of edge weights or similar.

As already described above, the videos may be ranked using a newly uploaded video as comparison video, which may serve as reference or sorting anchor. However, it is also possible to rank the gallery itself, i.e., rank all existing videos, timely independent from a video upload, analysis and/or evaluation. For example, all videos may be ranked or sorted based on the score “economy of motion” in descending order. This may be useful when users just want to glance through the gallery and search for surgery videos with specific properties, rather than aiming for comparing a single surgery against others.

In a further embodiment, instead of using an individual score value, an average score value of all videos may be calculated, and the videos may be ranked or sorted according to a deviation from this average score value. This may be used e.g., for identifying non-standard-performing personal, or non-standard-performing residents in the learning group. In this context, interesting scores may be for example refractive outcomes of the surgery, time of possible thermal impact on the cornea, duration of injector being operated compared to injector instruction recommendation, etc.

Further, it may be ranked according to a deviation from a given target score value. Such a ranking may be beneficial in case of a heavily skewed distribution of score values over different surgeons. In this case, sorting the video gallery by a deviation from a target score value (e.g., the desired surgery efficiency for a chain clinic) might be better suited.

Further, ranking may also be based additionally on meta-data (e.g., biometric data). In this case, a video may be compared with that of a similar surgery (e.g., similar pre-operative data like cataract stage, tools being used, etc.).

It should be noted that the above-described examples for ranking videos may also be used in combination. Also, variations, modifications and further developments are possible.

As described above, a user may view any videos from a video gallery, including the loaded video, for training purposes instead of only viewing the evaluation results, e.g., derived scores. A video gallery may allow a user to select videos for watching, to thereby learn e.g., how experienced surgeons handle tools more efficiently, how experienced surgeons handle exceptional situations, how experienced surgeons handle different devices or use different techniques to be overall more efficient, or safer, or both, etc. However, as explained above, video galleries grow over time, and it becomes more and more time consuming to find a good video for watching and learning. Although the ranking of videos in a video gallery provides an approach for finding videos which are interesting, a user may still know only after selection and after watching a video from the (ranked) gallery, if the selected video was what the user was actually interested in.

Therefore, according to a further embodiment, the feedback method comprises generating, based on the evaluation result, a summarizing video clip containing a portion of the original video data of one or more surgeries. In this context, a portion of the original video data is to be understood as more than one frame per summarizing video clip, but less frames than the complete video. The summarizing video clip may be for example a gif or a mini video clip. This is in contrast to a thumbnail image which consists of only one frame.

A single thumbnail image does not necessarily capture all the potentially relevant parts of a surgery. In consequence, a user might easily oversee a video which would have been helpful for learning, but which had a non-interesting thumbnail, e.g., a thumbnail could show the specific cataract before the surgery, but the user would be interested in videos of a specific phacoemulsification technique which is not shown by the thumbnail. In contrast to that, a summarizing clip may allow a user to qualitatively estimate the relevance of a surgery video's content before watching the whole video. The summarizing video clip may preferably reveal the relevant steps of the original video which may be presented to the user during the video selection as a preview. Since more than one frame is shown to represent a surgery, the chance of missing relevant parts may be significantly reduced, while it requires orders of magnitudes less time to watch the clip than watching the entire video.

According to a further embodiment, the feedback method further comprises extracting frames of the video data for generating the video clip. Preferably, the extracted frames contain events of interest of the video data.

These frames may be automatically calculated based on the evaluation as described above. For example, the events of interest may comprise key events during surgery which are most important to surgeons. The timestamp of these key events (e.g., phases, where specific tool is used, complication, etc.) can be estimated with machine learning and algorithmic solutions analyzing surgical workflow (phase segmentation, tool detection, metric calculation) as explained above.

In a further embodiment, more than one summarizing video clip may be created for a video. For example, a summarizing video clip may be created based on the scores/events of interest being present in the video. For instance, a summarizing video clip may be created for each score. The user may then select one or more scores for which the summarizing video clips should be displayed. In a further embodiment, the user may select a score and then the summarizing video clip may be created based on this selected score.

In the following, different variations of creating a summarizing video clip will be described. In one embodiment, the clip may be created based on central frame(s) per detected surgery phase. After analysis and evaluation of the video data, including segmentation of the video data into frames and detecting events of interest, in particular surgery phases, one or more central frames, e.g., the middle frame(s) of the corresponding surgery phase, may be selected and joined together to one summarizing video clip.

In a further embodiment, the video clip may be created based on at least one key frame per surgery phase. Instead of taking the central frame, a suitable key frame, e.g., showing the key feature of the surgery phase or being the most significant frame, may be selected for every surgery phase and the selected key frames may be joined together. This could also be used for comparison of two videos as will be described later.

In another embodiment, the clip may be created by selecting one or more frames in which a deviation from the standard surgery is recognized. In this case, especially the non-standard aspects of a surgery video are shown in the video clip. This may be for example the following deviations: incisions were recognized in unexpected order with other phases, extremely long rhexis was present, too long idle time during procedure was detected, etc.

According to a further embodiment, the clip may be created by selecting key frames without an explicit phase segmentation. In this embodiment, the creation of the summarizing video clip may take place during the analysis step by selecting meaningful frames for the entire video without the explicit knowledge due to the evaluation step.

According to a further embodiment, the feedback method further comprises removing frames of the video data and generating the video clip using the remaining frames. For example, the removed or omitted frames may correspond to frames which are similar or identical to preceding frames. For instance, the method may process the entire video, e.g., from start to end, and may iteratively add a new frame to the clip when the currently processed frame is visually different from all frames being part of the clip so far. In a further embodiment, creating the video clip may comprise initializing the clip to the full video, and then iteratively removing frames from the clip if a visually similar frame has already been kept earlier. Thereby, a clip may be obtained which represents all the different parts of the video, wherein phases and/or activities which occur at least twice will only be kept once.

Further, instead of selecting or removing frames, it may also be possible to add a flag or label to each frame whether it is part of the summarizing video clip or not. When playing the summarizing video, only the frames being marked as being part of the clip may be played whereas frames being marked as not being part of the clip may be skipped.

According to a further embodiment, the feedback method further comprises showing the summarizing video clip as a preview of the video data. As already described above, the summarizing video clip may be used for representing an overview of the content of the video to a user without the need to watch the complete video.

There may exist different options how the summarizing video clip may be shown, as will be explained in the following. It should be noted that any other way of presenting the summarizing video clip to a user is also possible.

For example, when the full video is in play-mode, a timeline may be shown which may be divided into different segments, each corresponding to a detected or recognized surgery phase. When hovering over the timeline, for example with a mouse, the corresponding frame of the summarizing video clip may be shown.

In one embodiment, the method may comprise switching between showing one image of the summarizing video clip as thumbnail and showing the entire summarizing video clip based on a user input.

For example, when viewing the video gallery, the gallery can show the thumbnail images, and when a user hoovers over the thumbnail, the corresponding video clip may be played. In a further embodiment, when viewing the video gallery, the gallery can show the thumbnail images, and when a user touches the part of the screen which displays the thumbnail, the corresponding clip may be played, and upon an additional touch playing may be stopped and the thumbnail may be shown again. Further, the gallery can show the thumbnail images, and play the clip of each one, one after the other, looping through all videos currently shown on the display unit. The clip can also be shown once the video is successfully uploaded and processed, as confirmation and summary of what was uploaded

In a further embodiment, key frames of the summarizing video clip may be used in a surgery report document. As described above, the evaluation results may be displayed or may be output otherwise, for example in the form of a report. In such a report, the frames of the whole video selected for the summarizing video clip can also be used in the text report of the surgery as a visual summary. For example, the generated report may conclude surgical events and immediate results of the surgery. The frames may be selected according to any one of the above-mentioned selection variations, in particular such that the report may contain relevant surgical moments in the summary report. The frames of the summarizing video clip may be listed as separate images when printing the report.

In a further embodiment, the user may select parts of the analysis video data, e.g., by giving a user input via a graphical user interface, and the video parts associated with the selected analysis video data may be joined together into a video clip and may than be outputted, e.g., by saving to disk and/or by saving in a database. As an example, a user may select some of the recognized phases in the video which shall be joined together into a video clip which may represent the video.

Although viewing surgery videos is beneficial for learning, surgery videos can be quite long, and surgeons might not have enough time to look through a whole video. In particular for learning, often only specific sub-parts of videos are relevant (e.g., to visually revisit the incision part). In addition, learning new skills or techniques can be done by comparing, e.g., on a qualitative level by watching the video of a very recent surgery and compare it with a video of a surgery performed by a different surgeon, with a different technique, or with different tools. Especially for the scenario of having two videos side-by-side, the efforts of finding the right sub-video for visually comparing are doubled and may be inefficient when done manually.

Therefore, according to a further embodiment, the feedback method comprises segmenting the video data into surgery phases and associating the surgery phases of the video data with the surgery phases of at least a second video. When displaying the videos, both videos may be displayed synchronously according to the associated surgery phases. For example, a user may select via a user input a surgery phase of both videos and the method may display the selected surgery phase in both videos simultaneously. In another embodiment, a specific surgery phase may be selected in only one of the videos and the other video may be displayed on a corresponding surgery phase, or at least at a similar surgery phase. When no corresponding surgery phase is present in the second video, the second video may be displayed at a timestamp at which such a surgery phase would normally take place, which will also be described below.

In contrast to manual forwarding of single videos or machine-learning based forwarding of single videos, as known from previous systems, the herein described feedback method provides the advantage to automatically jump to passages of the videos, for instance timestamps, as desired by a user. The timestamps may be automatically identified based on the statistical and machine-based analysis of the surgery video as described above. Further, not only a single video may be skipped, forwarded or the like to surgically relevant events, but two videos may be jointly skipped based on the user input for the first video. Also, other display modes may be possible.

Thereby, a solution for an efficient navigation in two videos may be realized, which further allows to select events of interest and/or scores and to relate the selected event of interest and/or score to the two corresponding sub-videos. In previous systems, this was only possible in an inefficient and iterative manual process, without clear relations between events of interest and/or scores and corresponding sub-videos.

Associating the surgery phases of two videos and/or jointly displaying the two videos may allow an efficient navigation in the two videos side-by-side, e.g., to quickly play specific sub-parts of the videos side-by-side for reference. When the video data is analyzed and/or evaluated as described above, the surgery phases are recognized, and this information may be also be used for displaying the two videos. Thus, no additional computation is necessary. Further, only one display may be needed for showing the two videos.

In a further embodiment, the videos being joined for instance to any one of the herein described examples may be combined to one video which can be stored for later displaying.

According to a further embodiment, the feedback method may comprise receiving a user input selecting an event of interest of the video, detecting the selected event of interest within the surgery phases of the video data, and detecting the selected event of interest or a surgery phase corresponding to the selected event of interest of the second video, and displaying the selected event of interest and/or the corresponding surgery phase in both videos simultaneously.

For a selected event of interest or score of the first video, there may be a directly corresponding event of interest or score in the second video. If not, a portion of the second video may be selected for displaying, during which the selected score or event of interest would typically occur. It may be preferred to select events of interest instead of general surgery phases so that frames which contribute to the in-depth analysis of the surgery are displayed as also described above.

Some examples of different navigations within the videos will be described in the following. It should be noted that other implementations are also possible and that the described examples may also be combined. For example, a user may select a specific score, e.g., incision attempts, for his/her video, i.e., the first video, jump the first video to a timestamp which is responsible for this specific score, and jointly jump the second video to the corresponding timestamp. The analysis and evaluation for defining the surgery phases, scores, and score values from both videos, e.g., “economy of motion”, “overall time”, “number of incisions”, etc., may be done before as described above. For example, the first video may be jumped to the beginning of the first incision phase and jointly the second video may be jumped to the beginning of the first incision phase.

In one example, both videos may be jointly jumped/skipped based on a specified surgery phase. That means that the two videos are jointly jumped to the corresponding first (or x-th) occurrence of a selected phase.

In another example, both videos are jointly jumped to a time stamp being the reason of a poor or good score value of the first and/or the second, reference video. For example, the first video is evaluated based on a selected score as described above. Both videos (first video and second reference video) jump to the time stamp which causes the poor score value (e.g., the frame where the first video has the eye most out-of-center).

In another example, instead of selecting the time stamp being the reason of a poor score value, the videos may be jointly jumped to a phase of the poor score or good value of the first and/or the second, reference video. Both videos (first video and reference video) may jump to the beginning of the phase associated with a poor score value (or a selected score), e.g., the frame of the incision phase which had a poor centration score value.

According to another example, both videos may jointly jump based on a tool presence (rather than a score value). In this case, the user may select as event of interest a tool of interest. Then, both videos may jump to the first usage of a particular tool which looks like one which the user selected from a list of available tools.

A further example may be to jointly jump to the occurrence of a clinically known abnormality. In this case, the event of interest may be a clinically relevant phenomenon (i.e., abnormal situation for standard cataract surgeries, e.g., the presence of an Argentinian flag phenomena). Both videos may jointly jump to the first detection of the selected phenomenon. If the phenomenon is not present in the reference video, a surgery phase may be selected during which such a phenomenon might occur. The detection of such a phenomenon or abnormality can be triggered based on available meta-data, such as text, comments, or the like, which describe the phenomenon.

According to a further example, both videos may jointly jump to shortcomings of the surgery detected by a machine learning model. For example, the machine learning model may identify the sequence causing a shortcoming in overall surgery metrics and both videos jump to that timestamp automatically. It may be possible to visually highlight the timestamp or/and the spatial region in which something may have gone wrong.

When the videos have not the same timeline, the method may further comprise adapting, i.e., stretching or extracting, a timeline of the at least two videos, or of one of the videos, such that the length of the surgery phases of the at least two videos correspond to each other. After jointly jumping both videos to the corresponding starting point, the user would usually start viewing both videos side-by-side. However, the reference video may show a superior technique, e.g., because a more experienced surgeon was chosen as reference, or because a more efficient technique was chosen as reference. In these cases, the overall time of the reference video may be shorter, whereas the user video may be longer. In this case, the shorter video can be slowed down, or the speed of the longer video can be accelerated, during playing such that the playing of both videos, and in particular the relevant passage, ends after the same physical time. E.g., if the reference video is twice faster than the user video, then the playing speed would be ½ for the reference video. This has also the advantage that the user may get a better impression of how much faster the reference technique is.

According to another example, a timeline of the first video including key-frames may be displayed. In some cases, only seeing a single frame of a surgery phase may be sufficient to assess the content of the phase, e.g., eye before intervention, capsular bag after phacoemulsification and polishing, intraocular lens after lens positioning. In consequence, the user could also be presented with a set of key frames extracted from the user video (as described above), select one, and then jump the video to this position, and the reference video to the visual-semantically closest frame. The key frames may be generated as described above in correspondence with the generation of a summarizing video clip.

According to a further aspect, a feedback system for surgeries, in particular eye surgeries, is provided. The feedback system comprises a processing device for loading video data from a surgery, for analyzing the video data, and for evaluating the analyzed video data, and an output device for outputting and/or displaying the evaluation result.

The feedback system may preferably be configured to perform the steps of the method for giving feedback on a surgery as being described above.

The features described with reference to the feedback method also apply to the feedback system. As also described above with respect to the feedback method, the different devices of the feedback system may be arranged at physically different locations. For example, the processing device may be implemented as part of a cloud server or may be implemented as one or more devices at several remote locations within a network.

For loading the video data from a surgery, the processing device may communicate with a storage unit, for example a database, being for example part of a cloud server, in which the video data is stored, as explained above. The communication between the processing device, the storage unit, and/or the output device may take place wireless, for example using any kind of radio communication network, or wired.

As described above with respect to the feedback method, the processing device may be one or more local computer (e.g., a clinical computer) or may be one or more server-based computer in a cloud computing service. Further, as described above, the processing device may be configured to execute the different steps of the feedback method physically decoupled (on several physically decoupled devices) and/or decoupled in time from each other, and/or to execute all steps on the same device and/or simultaneously.

The output device may be implemented as a local computer for outputting, e.g., printing, or displaying, for example on a connected display unit, the evaluation result. The devices may also be implemented as one single device. Such a display unit or display device may be any kind of display device being able to visualize the evaluation result, may also be a combined display and user input device, such as a touchpad, and/or may be any kind of user end device, such as a tablet or smartphone.

An even further aspect of the present invention relates to a computer program product comprising a computer program code which is adapted to prompt a control unit, e.g., a computer, and/or the processing device and the output device of the above discussed feedback system to perform the above discussed steps of the feedback method.

The computer program product may be provided as memory device, such as a memory card, USB stick, CD-ROM, DVD and/or may be a file which may be downloaded from a server, particularly a remote server, in a network, and/or may be accessed via and run in a web browser. The network may be a wireless communication network for transferring the file with the computer program product.

Further preferred embodiments are defined in the dependent claims as well as in the description and the figures. Thereby, elements described or shown in combination with other elements may be present alone or in combination with other elements without departing from the scope of protection.

In the following, preferred embodiments of the invention are described in relation to the drawings, wherein the drawings are exemplarily only, and are not intended to limit the scope of protection. The scope of protection is defined by the accompanied claims, only.

### BRIEF DESCRIPTION OF THE DRAWINGS

The figures show:

FIG. 1: a schematic block diagram of an exemplary system for giving feedback on a surgery;

FIG. 2: an exemplary flow diagram of a method for giving feedback on a surgery;

FIG. 3: an exemplary flow diagram of an embodiment of the method of FIG. 2;

FIGS. 4a-4b: examples of analysis results being determined by the method of FIG. 2 or 3;

FIG. 5: a first visualization example of the analysis of a surgery video using the method of FIG. 2 or 3,

FIG. 6: a second visualization example of the analysis of a surgery video using the method of FIG. 2 or 3,

FIG. 7: a third visualization example of the analysis of a surgery video using the method of FIG. 2 or 3, and

FIG. 8: a fourth visualization example of the analysis of a surgery video using the method of FIG. 2 or 3.

### DESCRIPTION OF EXEMPLARY EMBODIMENTS

In the following same or similar functioning elements are indicated with the same reference numerals.

As explained above, surgeries, in particular eye surgeries like cataract surgeries, or any other surgeries as mentioned above, are extremely complicated and require high skills to ensure an optimal surgery outcome that meets the expectations of a patient, for example regarding the visual acuity. To achieve the necessary skills, surgeons require intensive training before they become expert in the specific operating field. For this purpose, a feedback system and a corresponding feedback method may be used, which will be described with reference to the following figures.

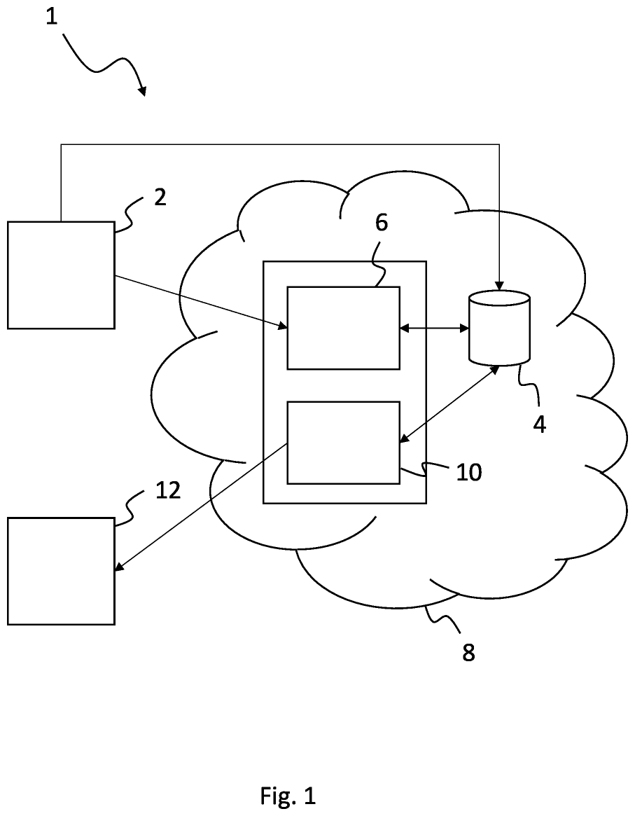

FIG. 1 shows a feedback system 1 for surgeries, in particular eye surgeries or any other surgery as mentioned above, which may be used for training surgeons regarding different kind of surgeries, in particular eye surgeries such as cataract surgeries. Before, during and after surgeries, video data may be generated from any device 2, for example from operation microscopes or the like. The video data may be uploaded to a database or storage unit 4 or may directly be uploaded to a processing device 6. As illustrated in FIG. 1, the feedback system 1 may be implemented as a cloud-based system, wherein the processing device 6, the database 4 and an output device 10 are implemented within a cloud 8 and the medical device 2 and a display unit 12 are remotely located. However, it should be noted that the different devices may also be implemented as one single device.

For providing a feedback to a surgeon, who has conducted a surgery, the device 2 may upload video data to the processing device 6 directly or via the data base 4. The video data may be provided in any kind of video file format, for example MPEG, and comprise multiple still images, i.e., frames, from the surgery. Each frame may show an image of a body part the surgeon is operating on and, optionally, may further comprise any kind of operating tool used by the surgeon. Optionally, also frames showing no surgery activity and/or no body part may be contained. Further, the video data may also comprise meta-data such as patient data or the like.

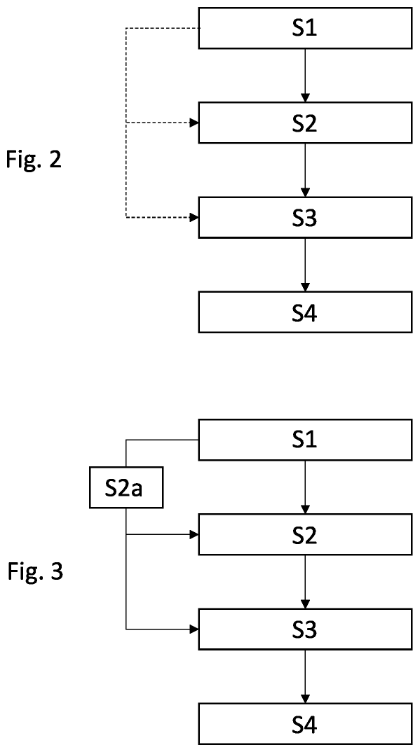

The video data may be processed within the processing device 6, in particular analyzed and evaluated as will be described in the following with reference to FIG. 2. The output device 10 may output the evaluation result, for example to the display unit 12, or may output a report in text form, for example printed. The display unit 12 may be any kind of display device being able to visualize the evaluation result, may also be a combined display and user input device, such as a touchpad, and/or may be any kind of user end device, such as a tablet or smartphone.

The analysis and evaluation of the video data will be described in the following with reference to FIG. 2 illustrating a corresponding feedback method and FIG. 3 illustrating an exemplary embodiment of the feedback method of FIG. 2.

In a first step S1 of the feedback method, the video data is received and/or loaded. Then, in a subsequent step S2, the video data may be analyzed. Analyzing in this context may refer to any kind of processing of the video data which is suitable to provide information about the video data, for example about the content. During analysis of the video data, the video data may be processed, resulting in analyzed video data. The analyzed video data may be for example video data being segmented or being examined regarding the content or additional information like meta-data.

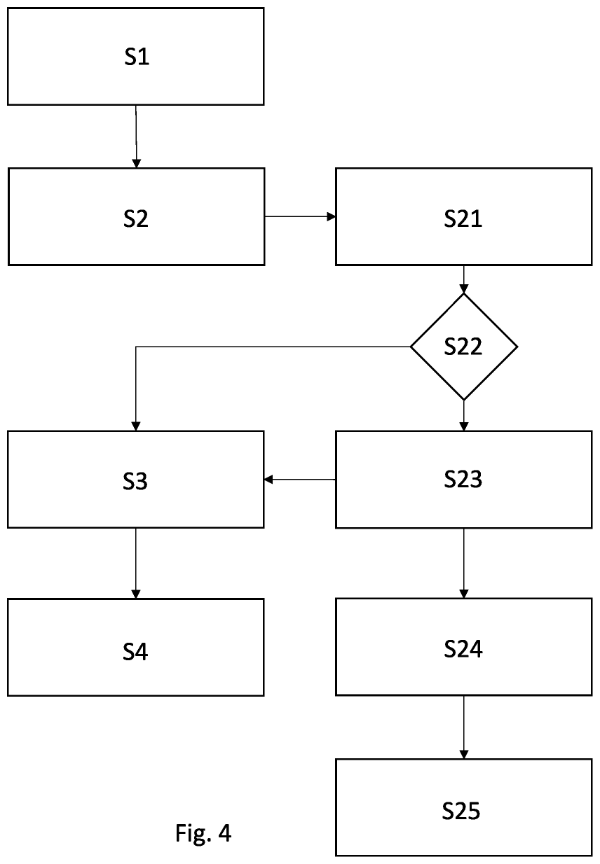

The video data may include at least one video file having multiple frames 14 (as shown in FIG. 4a). The video data may further comprise meta-data, such as pre-surgery or post-surgery data, patient data and/or recording data from medical devices.

In step S2, temporal and/or spatial semantic video segmentation, object detection, object tracking and/or anomaly detection may be performed. The information, which are gathered by this processing, may be referred to as meta-representations or analyzed video data.

When analyzing the video data, the multiple frames 14 of the video data may be segmented. The analysis of the video data may comprise a temporal and/or spatial semantic frame segmentation 15 (as for example shown in FIG. 4b). This semantic frame segmentation may be carried out frame by frame (temporal semantic frame segmentation), and/or every pixel of a frame may be assigned to its semantic category (spatial semantic frame segmentation), in this case anatomical parts of a human eye 16 as well as a tool 17. The analysis of the video data may further comprise an anomaly detection. In this case, it may be determined for every frame if an unknown event is happening. Anomaly detection may also be carried out on a pixel level, for tools, etc. Furthermore, the analysis of the video data may comprise object detection and tracking 18 within the video data (as shown in FIG. 4c). Thereby, individual tools may be localized in every frame using bounding boxes 19 with trajectories between the several frames and their trajectory 19 may be computed over time. The localization of the tools, i.e., the object detection, may be combined with the semantic frame segmentation.

In a next step S3, this information can be used for evaluating the analyzed video data, for example for providing any information to a user regarding an assessment of the corresponding surgery. Evaluating the analyzed video data includes detecting at least one event of interest within the video data and/or deriving at least one score from the at least one event of interest. As described above in detail, events of interest may be any feature or characteristic of the surgery which can be used for indicating important processes within the surgery.

For each derived score, a score value may be determined during the evaluation. The score value may be determined as an absolute value, for example absolute number of incision attempts, phase length, etc. Alternatively, the derived score may be compared with data being stored e.g., in the database 4. In this case, the score value may provide an assessment of the currently conducted surgery compared with a reference value, e.g., from a previous surgery, for example of a highly skilled surgeon.

As mentioned above, an event of interest may be for example a specific surgery phase. When comparing the length (score) of this surgery phase of the current surgery with the length (score) of a surgery phase of a preceding surgery, a score value may be derived which indicates how good in terms of speed the current surgery, i.e., the surgeon, has been compared with other surgeries or surgeons. Alternatively, the absolute length of the surgery phase may directly be used as score value. Further score values may be for example the maximum length of all surgery phases from a specific phase type during a single surgery (e.g., the longest incision phase if multiple incisions have been conducted during one surgery), the minimum length of a surgery phase, focus during the surgery with regard to patient eye, number of enters/exits of instruments in the eye, etc.

The steps S2, S3 of analyzing the video data and evaluating the analyzed video data may also be combined or merged or may be performed more or less simultaneously. Further, analyzing the video data S2 and/or evaluating the analyzed video data S3 can be carried out using a machine learning algorithm. For example, video data, analysis results and/or evaluation results from previous surgeries may be used as training data sets. Further, machine learning algorithms may be implemented for example using neural networks and/or may be implemented as self-learning algorithms so that they can be trained, or fine-tuned, continuously during the analysis and/or evaluation of video data. An example of refining the feedback method including a machine learning algorithm used therein will be described below with reference to FIG. 3.

After the evaluation, an evaluation result may be output in step S4 and may be for example displayed on the display unit 12. Further, the evaluation result may be integrated into a report, for example into text, and may optionally be printed. The analysis and/or evaluation result may be displayed in several variations and may be used for different further purposes which will be described below.

As already explained, the feedback method may further comprise refining the analysis and/or evaluation based on a user input as shown in FIG. 3. This may further improve the accuracy of the analysis and/or evaluation in steps S2, S3. For example, the user input may verify and/or correct the analyzed video data. Correcting in this context may refer to a confirmation or rejection of the analyzed video data.

An exemplary feedback method may thus load the video data S1 and analyze the video data S2 as described above, e.g., including frame segmentation, tool tracking, etc. Subsequent to the analysis, the user may review the analyzed video data in step S21. For example, the user verifies in step S22 whether a tool was tracked correctly, whether the semantic categories of the frames are correctly identified, whether an anomaly was detected correctly etc.

If everything was analyzed correctly, the user confirms the analysis in step S22, and the method continues with step S3 as described above.

If the user rejects the analysis in step S22, the user may correct or refine the analysis in step S23. This may be done via any suitable user interface. After refining or correcting the analysis, the method continues with step S3 as described above.

In addition, the verified or corrected analyzed video data of step S23 may be used for training of a machine learning algorithm S25. In this case, the machine learning algorithm receives the corrected and/or verified analyzed video data as input training data. The machine learning algorithm may then use the corrected information when analyzing video data in step 2. Over time, this may reduce the efforts for verifying and correcting the analyzed video data as the machine learning algorithm becomes better and better.

In a further optional step S24, which can take place before step S25 and after step S23, the correction of the user may be verified and may even be corrected further. This may further contribute to the training of the machine learning algorithm and/or may improve the performance of the machine learning algorithm, as only verified corrected analyzed data are used for training.

As mentioned above, the evaluation result may be used for different purposes, all of them giving the surgeon or user a feedback on a surgery. Some examples will be listed below. It should be noted that they can be implemented as single use cases or may be combined.

In one embodiment, a learning progress of a user and/or a user group can be tracked based on the evaluation result, in particular over multiple surgeries. Thus, the user may get a feedback not only to one conducted surgery but to multiple surgeries and his/her development over the multiple surgeries. In this case, the evaluation result of one surgery may be compared with at least one further surgery of the same user and the learning progress may be tracked based on a result of the comparison. For example, the learning progress may be determined by comparing one or more score values of one surgery with the corresponding score values of a preceding surgery. If the score values are getting better, the surgeon is considered to make a learning progress. This may be the case for decreasing score values when a small value of the score indicates a good performance, or for increasing score values when a high value of the score indicates a good performance. The determined and achieved learning progress may be displayed by the display unit 12.

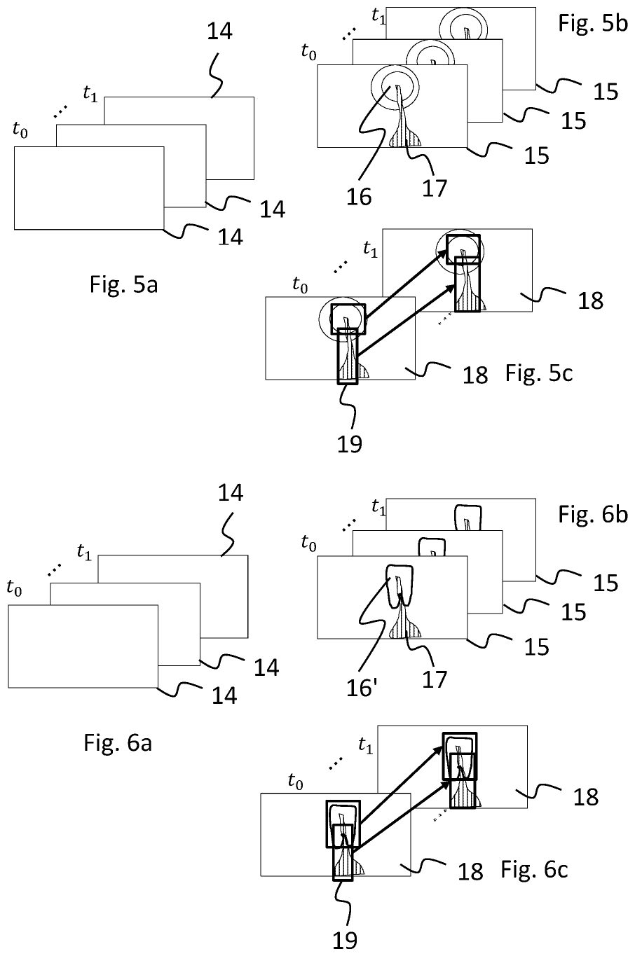

One example of a visualization of such a learning progress is shown in FIG. 5. On the y-axis, a score value may be used as reference and on the x-axis, the time (either as absolute time or as number of surgeries) can be shown. In addition to the tracked learning progress, e.g., the scores derived from conducted surgeries (named as surgery efficiency), shown by the continuous line, a prediction of the learning progress may be visualized (surgery efficiency est.), shown by the dashed line.

In the database 4, a plurality of videos can be stored over time. A surgeon can use the videos for learning or training. A good learning experience can for example be achieved by visually comparing two videos, e.g., the video of the recent surgery with a reference video.

First of all, a surgeon or other user might need to select a video. Finding a suitable reference video in a large and growing video gallery is very complex, which may delay or hinder the learning success. Therefore, the described evaluation result or score may be used for ranking the plurality of videos. For this purpose, the evaluation result of one video may be compared with evaluation results of one or more other videos and a rank of each of the multiple videos may be determined based on the comparison.

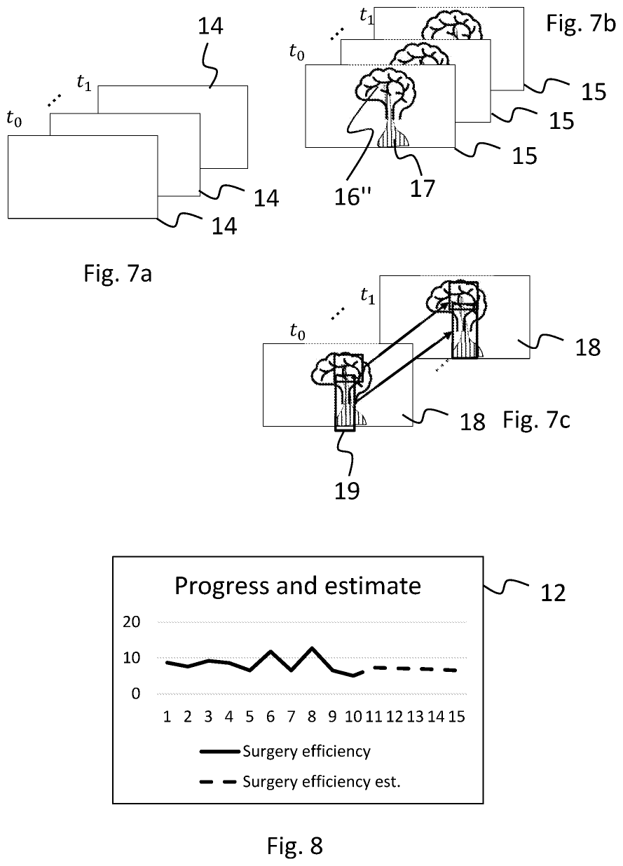

FIGS. 6 and 7 show two possible visualizations for selecting ranking criteria. As shown in FIG. 6, on the display unit 12, a list 20 of ranking criteria may be provided, selectable by the user as check boxes 22. If the list 20 is longer than can be shown on the display unit 12, a sliding bar 24 may be used for scrolling through the list 20. Further, in this example, a minimum length and maximum length 26 of the video may be selected. When the user has selected the ranking criteria, the ranking may be started by activating the sort button 28.

Another visualization example is shown in FIG. 7. In contrast to FIG. 6, the display unit 12 shows together with the selection choices for the ranking the current video 30. Further, it is possible to have for some of the ranking criteria an additional drop-down menu 32. Such a drop-down menu 32 may be used for each criterion which may have sub-selection possibilities.

Such ranking of videos leads to an order of the videos such that related videos are grouped together, based on the evaluation results. Videos, in which similar events of interest are recognized, or which have similar or identical scores or score values, may be grouped together or at least close to each other. After ranking, the videos may be displayed in a gallery representation on the display unit 12 based on the determined rank of the videos. The gallery representation may be any kind of suitable representation, for example depending on the used display unit 12. For example, the videos may be sorted in ascending or descending order, based on the comparison result. Preferably, the videos may be sorted according to a user choice, for example based on the score incision attempts. For example, several videos may be shown next to each other in one row, e.g., three videos, and there may be further rows with several videos, displayed beneath each other. Other representations are also possible.

When showing the videos in a gallery representation, the videos may be shown using a summarizing video clip. Such a video clip may allow a user to have a short overview of the video to see if the selected video is what the user was actually interested in.

Such a summarizing video clip contains a portion of an original video, for example may reveal the relevant steps of the original video which may be presented to the user during the video selection as a preview. Since more than one frame is shown to represent a surgery, the chance of missing relevant parts may be significantly reduced. There exist different ways of creating a summarizing video clip. For example, the clip may be created based on central frame(s) per detected surgery phase or by selecting one or more frames in which a deviation from the standard surgery is recognized. When viewing the video gallery, the gallery can show thumbnail images of the videos, and when a user hoovers over the thumbnail, the corresponding clip may be played, and when the user hoovers away from the clip, the clip is stopped ant the thumbnail is shown again or the last shown frame from the clip is shown as still thumbnail.

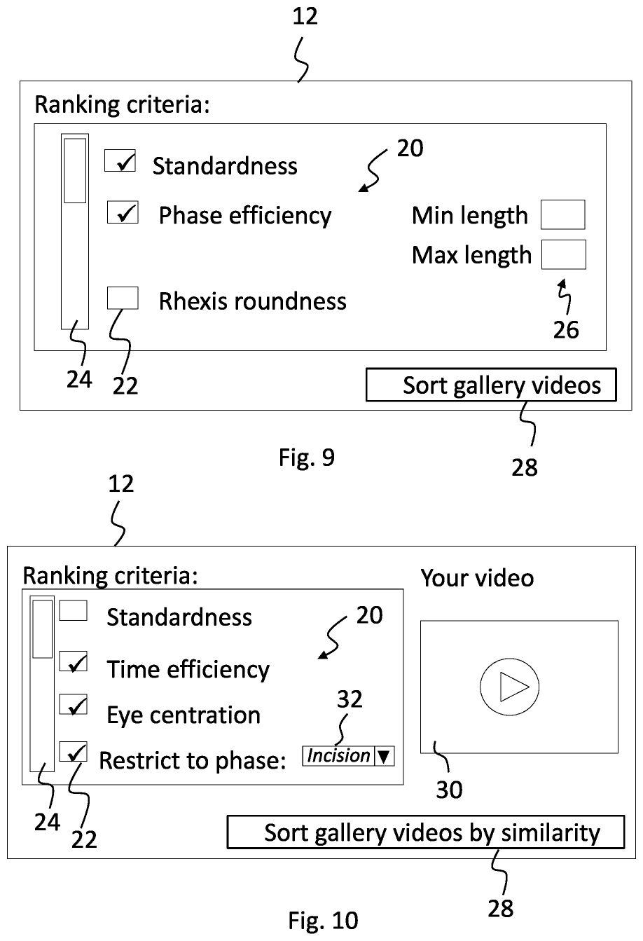

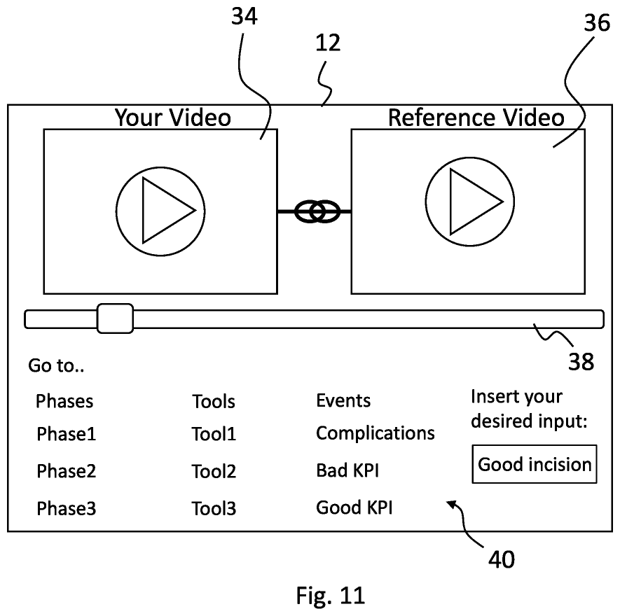

In order to improve learning, a user or surgeon may directly compare two videos with each other, for example one video of a newly conducted surgery and a reference video. For this purpose, two videos may be associated with each other and may be displayed together as shown in FIG. 8.

On the display unit 12, two videos 34, 36 may be played side-by-side. The first video 34 is a video of a newly conducted surgery and the second video 36 is a reference video, for example of a skilled surgeon. The analysis and evaluation as described above may be used for associating surgery phases of the videos 34, 36 with each other. When displaying the videos 34, 36, both videos may be displayed synchronously according to the associated surgery phases as shown by the one timeline 38. Associating the surgery phases of the two videos 34, 36 and jointly displaying the two videos 34, 36 may allow an efficient navigation in the two videos side-by-side.

As can be seen in FIG. 8, a user may have several selection choices 40 for selecting a surgery phase, for example using the scores or score values being determined as described above. When moving within the first video 34, the second video 36 will be moved accordingly. Thus, a user may select a specific surgery phase, score, or the like for the first video 34 and the second video 36 will be played at the associated timestamp, showing the same surgery phase or score or a timestamp at which such a surgery phase would normally take place.

In addition to the herein described use cases for the analysis and evaluation of surgery video data, other use cases and implementations are possible. Also, the described embodiments may be combined when suitable.

In summary, using the above-described feedback system and method, it is possible to provide a machine-based, human-independent analysis and evaluation of surgery video data. In particular the evaluation result may be used as training feedback for a surgeon by giving information about the performed surgery the video data originates from.

### REFERENCE NUMERALS

1 feedback system
2 (medical) device
4 database
6 processing device
8 cloud
10 output device
12 display
14 frames
15 semantically segmented frames
16 eye
17 tool
18 object detected and tracked frames
19 bounding boxes with trajectories
20 ranking criteria
22 check boxes
24 sliding bar
26 length
28 sort button
30 video
32 drop-down menu
34 first video
36 reference video
38 timeline
40 selection choices
S1-S4 method steps
S21-S25 method steps

1-47. (canceled)

48. A method for giving feedback on a surgery, in particular an eye surgery, the feedback method comprising the steps of:
loading and/or receiving video data from a surgery by a processing device,
analyzing the video data by the processing device,
evaluating the analyzed video data by the processing device, and
outputting and/or displaying the evaluation result by an output device,
further comprising the step of tracking, by the processing device, a learning progress of a user and/or a user group based on the evaluation result of multiple surgeries.

49. The method according to claim 48, further comprising the step of predicting, by the processing device, a development of the learning progress based on the tracked learning progress and/or stored learning progresses of other users.

50. The feedback method according to claim 48, further comprising the step of detecting and/or tracking of at least one region of interest within at least one image of the video data.

51. The feedback method according to claim 50, further comprising the step of reducing and/or weighting the content of the video data based on the detected region of interest.

52. The feedback method according to claim 48, wherein the steps of analyzing the video data, and/or evaluating the analyzed video data and/or detecting and/or tracking the at least one region of interest is carried out by at least partially using a machine learning algorithm.

53. The feedback method according to claim 48, wherein the step of analyzing the video includes at least phase segmentation.

54. The feedback method according to claim 48, wherein the step of analyzing the video includes at least object detection and/or object tracking.

55. The feedback method according to claim 48, wherein the step of analyzing the video includes at least spatial semantic video segmentation.

56. The feedback method according to claim 48, wherein the step of analyzing the video data includes deriving at least one score value for at least one defined score and/or event of interest and/or region of interest directly from the video data by using a machine learn algorithm.

57. The feedback method according to claim 48, wherein the step of evaluating the analyzed video data includes detecting at least one event of interest within the video data and/or detecting at least one region of interest and deriving at least one score from the at least one event of interest and/or deriving at least one score from the at least one region of interest.

58. The feedback method according to claim 57, wherein the at least one event of interest is a specific surgery phase and wherein the at least one score derived from the specific surgery phase is a frequency of the surgery phase or a length of the surgery phase.

59. The feedback method according to claim 57, wherein the at least one event of interest is a presence of a tool and wherein the at least one score derived from the presence of the tool, includes at least one of: a relative position of the tool, an absolute position of the tool, and a speed of the tool.

60. The feedback method according to claim 57, wherein the step of evaluating the analyzed video data further comprises determining a score value for the at least one score, wherein the score value is a quality of instrument handling, a speed value of a speed of the tool, an absolute length of the surgery phase and/or a relative length of the surgery phase compared with a length of surgery from another surgery.

61. The feedback method according to claim 57, further comprising the step of comparing the at least one score with stored data, in particular with historical data of previous surgeries, wherein determining a score value of the at least one score is based on the comparison result.

62. The feedback method according to claim 48, further comprising the step of visualizing the evaluation result, using at least one of: bars, pie charts, trend graphs, overlays.

63. The feedback method according to claim 48, further comprising the step of comparing the evaluation result of one surgery with at least one further surgery of the same user and tracking the learning progress based on a result of the comparison.

64. The feedback method according to claim 48, wherein the display unit is configured to display the learning progress.

65. The feedback method according to claim 49, further comprising the step of estimating a time until when a specific learning level, in particular a specific score value, will be reached based on the predicted development.

66. The feedback method according to claim 65, further comprising the step of receiving a user input defining the specific learning level.

67. A feedback system for surgeries, in particular eye surgeries, the feedback system comprising
a processing device for loading and/or receiving video data from a surgery, for analyzing the video data, for evaluating the analyzed video data, and for tracking a learning progress of a user and/or a user group based on the evaluation result of multiple surgeries, and
an output device for outputting and/or displaying the evaluation result,
wherein the feedback system is configured to perform the steps of the feedback method according to claim 48.
